(19) United States
(12) Patent Application Publication
(10) Pub. No.: US 2025/0195296 A1
(43) Pub. Date: Jun. 19, 2025

(54) ABSORBENT ARTICLE HAVING ONE OR MORE LAYERS INCLUDING CELLULOSIC FIBERS

(71) Applicant: The Procter & Gamble Company, Cincinnati, OH (US)

(72) Inventors: Nadia KURBATOVA, Hessen (DE); Clarissa MALDONADO, Cincinnati, OH (US); Gerard Alain VIENS, Wyoming, OH (US); Ezgi KECELI, Frankfurt (DE); Carla KRELL, Hessen (DE); ImkeAnna HOHN, Schwalbach am Taunus (DE); Caroline CARRIEL SCHMITT, Schwalbach am Taunus (DE)

(21) Appl. No.: 18/978,102

(22) Filed: Dec. 12, 2024

Related U.S. Application Data
(60) Provisional application No. 63/611,269, filed on Dec. 18, 2023.

(51) Int. Cl. A61F 13/53; A61F 13/511; A61F 13/56

(52) U.S. Cl. CPC A61F 13/53; A61F 13/51121; A61F 13/5605

(57) ABSTRACT

An absorbent article that may be used as a feminine hygiene product, such as liner. The absorbent article may include at least one of a topsheet layer, a fluid management layer, an absorbent core layer, a first barrier layer, and a second barrier layer. Each layer included in the absorbent article may include cellulosic material, such as cotton and rayon. Each of the layers of the absorbent article may be void of synthetic fibers and films and/or substantially free of synthetic materials.

### CROSS REFERENCE TO RELATED APPLICATIONS

This application claims the benefit, under 35 U.S.C. § 119 (c), to U.S. Provisional Application No. 63/611,269, filed Dec. 18, 2023, the entire disclosure of which is fully incorporated by reference herein.

### FIELD OF THE INVENTION

The present disclosure relates to disposable absorbent articles, more specifically disposable feminine hygiene absorbent articles, including one or more layers that are void of synthetic fibers and films.

### BACKGROUND OF THE INVENTION

Absorbent sanitary articles are used to collect various bodily fluids for hygiene purposes. For example, pantiliners, or liners, are normally used by women to receive and contain discharges, including menstrual fluid, vaginal discharge, and urine in the case of incontinence. Consumers often find it desirable to wear liners for long periods of time and on a daily or otherwise frequent basis. Because the use of liners is daily or otherwise more frequent than other types of absorbent articles, consumers are becoming increasingly concerned over the number of liners they discard and the implications of such on the environment. Traditionally, liners have been made solely or predominately with synthetic materials, such as synthetic fibers and films. Examples of these synthetic fibers and films include polyethylene, polypropylene, and other plastic materials. Generally, these synthetic fibers and films remain in the environment for prolonged period of time and/or require intervention to be collected and re-used or to be broken down.

However, switching from largely synthetic materials to more natural materials, such as cellulosic materials, presents issues in terms of wearer comfort and product performance. For example, cellulosic material, such as pulp, is known to be adequately absorbent but may lack the structural integrity to be wetted and worn in a relatively moist environment for long periods of time. Further, cellulosic materials are generally more porous and hydrophilic, which may present issues in terms of providing an adequate barrier for fluid. Additionally, cellulosic materials may be more costly and present issues for high speed manufacturing due to their properties.

Therefore, there remains a need to provide an absorbent article with cellulosic materials that provides adequate absorption, dryness, resiliency, and comfort. Further, there is a need to provide desired properties in a cost efficient and effective manner.

### SUMMARY OF THE INVENTION

An absorbent article may include: a liquid pervious topsheet layer comprising cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises cellulosic material comprising fibers having from about 1.3 to about 10 decitex; a first barrier layer comprising at least one of cellulosic fibers and cellulosic film; a second barrier layer disposed adjacent to the first barrier layer, wherein the second barrier layer comprises cellulosic material; and an absorbent core layer disposed between the fluid management layer and the first barrier layer. The absorbent article has an acquisition time of less than about 1 second and a wet through of less than about 0.3 g. Additionally, the topsheet layer, the second barrier layer, the fluid management layer, the first barrier layer, and the absorbent core layer are void of synthetic fibers and films.

An absorbent article may include: a liquid pervious topsheet layer comprising a cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising carded staple fibers, wherein the carded staple fibers have from about 1.3 to about 10 decitex, and wherein the staple carded staple fibers have an area moment of inertia of from about 2,000 μm⁴ to 95,000 μm⁴; a second barrier layer adjacent to the fluid management layer, wherein the second barrier layer comprises a cellulosic material; and an absorbent core layer disposed between the fluid management layer and the second barrier layer. The absorbent article is substantially free of synthetic fibers and films.

An absorbent article may include: a liquid pervious topsheet layer comprising a cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising fibers having from about 1.3 to about 10 decitex; a first barrier layer comprising at least one of cellulosic fibers and cellulosic film, wherein the first barrier layer has a repellency of at least 5 seconds and a 10% of Pore less than about 20 microns; a second barrier layer adjacent to the first barrier layer, wherein the second barrier layer comprises cellulosic material; and an absorbent core layer disposed between the fluid management layer and the first barrier layer. The first barrier layer and the fluid management layer are void of synthetic fibers and films.

### BRIEF DESCRIPTION OF THE DRAWINGS

FIG. 1 is a perspective view of a nonlimiting example of an absorbent article in the form of a liner.

FIG. 2A is an exploded view of the absorbent article in the form of a liner.

FIG. 2B is an exploded view of the absorbent article in the form of a liner.

FIG. 3 is a schematic cross sectional representation an exemplary absorbent article having layers.

FIG. 4 is a schematic cross sectional representation an exemplary absorbent article having layers.

FIG. 5 is a schematic cross sectional representation an exemplary absorbent article having layers.

FIG. 6 is a schematic cross sectional representation an exemplary absorbent article having layers.

FIG. 7 is a schematic representation an exemplary creping process.

FIG. 8A is a top view of a plurality of absorbent articles progressing in a machine direction.

FIG. 8B is a top view of a plurality of absorbent articles progressing in a machine direction.

FIG. 9 is an exploded view of an absorbent article including various layers and adhesives.

FIG. 10 is an exploded view of an absorbent article including various layers and adhesives.

FIG. 11A is a perspective view of a portion of an individual fiber.

FIG. 11B is a cross sectional view of a solid individual fiber and the equation for the area moment of inertia of such a fiber.

FIG. 11C is a cross sectional view of a hollow individual fiber and the equation for the area moment of inertia of such a fiber.

FIG. 12 illustrates an example graph that depicts the three curves with the points of interest marked as described in the Capillary Flow Porometry test method.

FIG. 13 illustrates a schematic representation of a testing mechanism comprised of a box used in determining the sound.

FIG. 14 illustrates a schematic representation of a rotational mechanism used in determining the sound.

FIG. 15A illustrates a schematic representation of an ultra sensitive fixture used in the Three Point Bend test method.

FIG. 15B illustrates a schematic representation of an ultra sensitive fixture used in Three Point Bend test method.

FIG. 15C illustrates a schematic representation of an ultra sensitive fixture used in the Three Point Bend test method.

### DETAILED DESCRIPTION OF THE INVENTION

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene articles.

“Design element” as used herein means a shape or combination of shapes that visually create a distinct and discrete form, regardless of the size or orientation of the form. Design elements may comprise objects, character representations, words, colors, shapes or other indicia that can be used to distinguish, identify or represent the manufacturer, retailer, distributor and/or brand of a product, including but not limited to trademarks, logos, emblems, symbols, designs, figures, fonts, lettering, crests or similar identifying marks. Design elements and/or combinations of design elements may comprise letters, words and/or graphics such as flowers, butterflies, hearts, character representations and the like. Design elements and/or combinations of design elements may comprise instructional indicia providing guidance or instruction relative to placement and/or fit of the article about the wearer.

“Feminine hygiene article” refers to disposable absorbent articles to be worn by women for menstrual and/or incontinence control or for vaginal discharge. These articles are commonly referred to as pads, pantiliners/liners, sanitary napkins or sanitary towels. These articles have generally flat surfaces and are typically held in place adjacent the user's crotch (i.e., the pubic region) by the user's undergarment. Feminine hygiene articles can be placed into the user's undergarment and affixed via adhesive or other joining means.

The term “integrated” is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing uses a plurality of high pressure water jets to entangle fibers. Needlepunching involves the use of needles to push and/or pull fibers to entangle them with other fibers in the nonwoven.

The “longitudinal” direction is a direction running parallel to the maximum linear dimension. “Length” of the article or component thereof, when used herein, generally refers to the size/distance in the longitudinal direction.

The “lateral” or “transverse” direction is orthogonal to the longitudinal direction, i.e., in the same plane of the majority of the article and the longitudinal axis, and the transverse direction is parallel to the transverse axis. “Width” of the article or of a component thereof, when used herein, refers to the size/distance of the dimension orthogonal to the longitudinal direction of the article.

“Film” means a sheet-like material wherein the length and width of the material far exceed the thickness of the material (e.g., 10×, 50×, or even 1000× or more). Films are typically liquid impermeable but may be configured to be breathable.

The “Z-direction” is orthogonal to both the longitudinal and transverse directions.

“Machine Direction” or “MD” as used herein means the direction parallel to the flow of the material through the material making machine and/or absorbent article product manufacturing equipment.

“Cross Machine Direction” or “CD” as used herein means the direction parallel to the width of material making machine and/or absorbent article product manufacturing equipment and perpendicular to the machine direction.

As used herein “hydrophilic” and “hydrophobic” have meanings as well established in the art with respect to the contact angle of water on the surface of a material. Thus, a material having a water contact angle of greater than about 90 degrees is considered hydrophobic, and a material having a water contact angle of less than about 90 degrees is considered hydrophilic. Compositions which are hydrophobic, will increase the contact angle of water on the surface of a material while compositions which are hydrophilic will decrease the contact angle of water on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material.

As used herein, the term “nonwoven web” refers to a web having a structure of individual fibers or threads which are interlaid, but not in a repeating pattern as in a woven or knitted fabric, which do not typically have randomly oriented fibers. Nonwoven webs or fabrics have been formed from many processes, such as, for example, meltblowing processes, spunbonding processes, hydroentangling, needlepunching, airlaying, and bonded carded web processes, including carded thermal bonding and carded air-through bonding.

### Absorbent Article

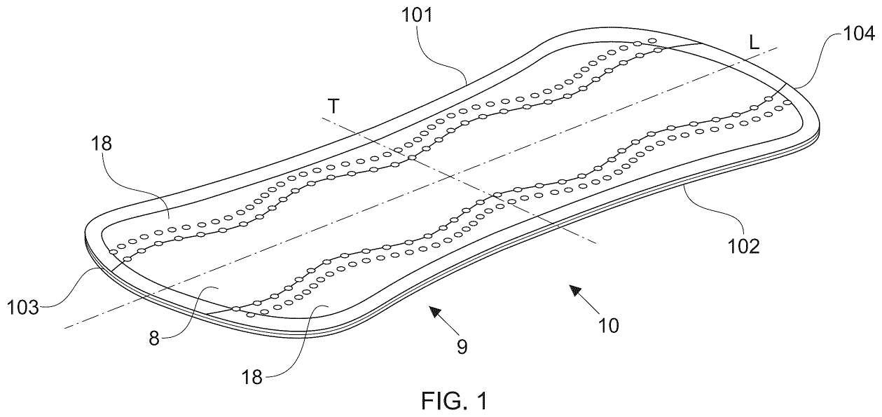

Absorbent articles are produced with a number of different absorbencies to provide various options based on the consumer's desired level of protection. One particular class of absorbent articles is referred to as a pantiliner or a liner, as these terms are used interchangeably. Liners are typically configured to be used when the user's needs are from about 1 mL to about 3 mL, which is relatively light amount in comparison to a user's needs when selecting a regular pad and/or a pant-type incontinence absorbent article. Liners may be configured to absorb menstrual fluid and/or other vaginal discharge, and, in some instances, to act as an additional layer of protection or perceived protection. Liners may be used by consumers, also referred to herein as users, for daily protection or reassurance. Liners of the present disclosure are design to accommodate an absorbent capacity of less than about 12 g or less than about 12 g or less than about 10 g of fluid according to the Dunk Capacity test method disclosed herein. FIG. 1 illustrates an example embodiment of an absorbent article 10, which is a liner. The absorbent article 10 includes a wearer-facing surface 8 in a typical pantiliner configuration. The absorbent article typically has a generally flat wearer-facing surface 8 but may be curved to conform to the user's body. For example, the wearer-facing surface 8 may include one or more protrusions that extend generally in a z-direction. The absorbent article 10 is relatively flexible to adapt to the user's anatomy. The article includes longitudinal sides 101, 102 and lateral ends 103, 104, any of which may be straight or curved. Each of the longitudinal sides extend in a direction generally parallel to a longitudinal axis L of the absorbent article. Each of the lateral ends 103, 104 extend in a direction generally perpendicular to the longitudinal axis L of the absorbent article. The absorbent article 10 includes a wearer-facing surface 8, which is opposite to the garment-facing surface 9. At least one of the garment-facing surface 9 and the wearer-facing surface 8 may include one or more design elements 18 such as indicia and/or tactile indicators.

Liners are generally smaller and more compact than pads. The caliper, also referred to herein as thickness, of the liners generally ranges from about 0.75 mm to about 20 mm, according to the Caliper test method disclosed herein. More specifically, liners may have a caliper of less than about 6 mm, or less than about 4 mm, or less than about 2.5 mm or less than about 1.5 mm or less than about 0.75 mm, according to the Caliper test method disclosed herein. Liners may have a caliper of from about 0.5 mm to about 6 mm or from about 0.75 mm to about 6 mm or from about 1 mm to about 4 mm, according to the Caliper test method disclosed herein. For example, liners may have a caliper of about 1.9 mm or about 2 mm or about 3 mm, according to the Caliper test method disclosed herein. The dimensions of the articles of the invention are adapted for the use intended as in known in the art.

These absorbent articles may have a longitudinal length measured substantially parallel to the longitudinal axis from the outermost point of the first lateral end 103 to the outermost point of the second lateral end 104. The longitudinal length of the absorbent article along the longitudinal axis L of the absorbent article may typically be between 10 cm and 25 cm, more typically between 12 cm and 21 cm or 15 cm to 19 cm. The absorbent articles may have a width measured substantially parallel to the transverse axis T from the outermost point of the first longitudinal side 101 to the outermost point of the second longitudinal side 102. The width of the absorbent article along the transverse axis T may typically be between about 3 cm and about 10 cm or between about 4 cm and about 7 cm or from about 6-7 cm. The total surface area of the wearer facing surface of a liner is between about 50 cm² and 150 cm², or from about 50 cm² to about 105 cm², or from about 50 cm² to about 80 cm². A typical surface area for a liner may be around 100 cm². These dimensions are merely indicative and not limiting because the dimensions of these and other types of absorbent articles may differ according to the intended use, as is known in the art.

The absorbent article 10 may include one or more design elements 18 disposed on at least one of the wearer-facing surface 9 and the garment-facing surface 8. The design elements 18 may be disposed on the wearer-facing surface 9 of the liner such that they are visible to the user. The design elements 18 may include one or more indicia and/or tactile indicators, such as emboss patterns. The absorbent article may also include indicia that communicates features of the absorbent article to the consumer, such as absorbency, orientation, and use. For example, an absorbent article may include indicia highlighting the areas of increased absorbency for the user or of channel(s) or embossment(s) placed for fluid handling. Examples of absorbent articles including indicia are described in U.S. Pat. Nos. 9,693,913 and 8,039,685, which are incorporated herein by reference.

Absorbent articles are usually substantially symmetrical in relation to the longitudinal axis L, such that the longitudinal axis divides the article in two substantially symmetrical halves (notwithstanding possible decorations such as embossments or printed patterns). However, if the absorbent article includes wings, these wings may be offset such that the absorbent article is not symmetrical about the longitudinal axis and/or the transverse axis.

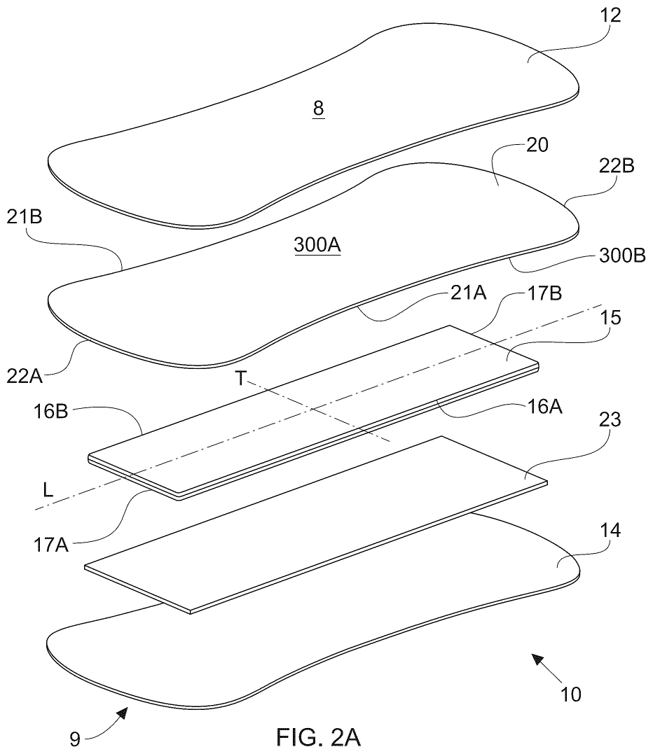

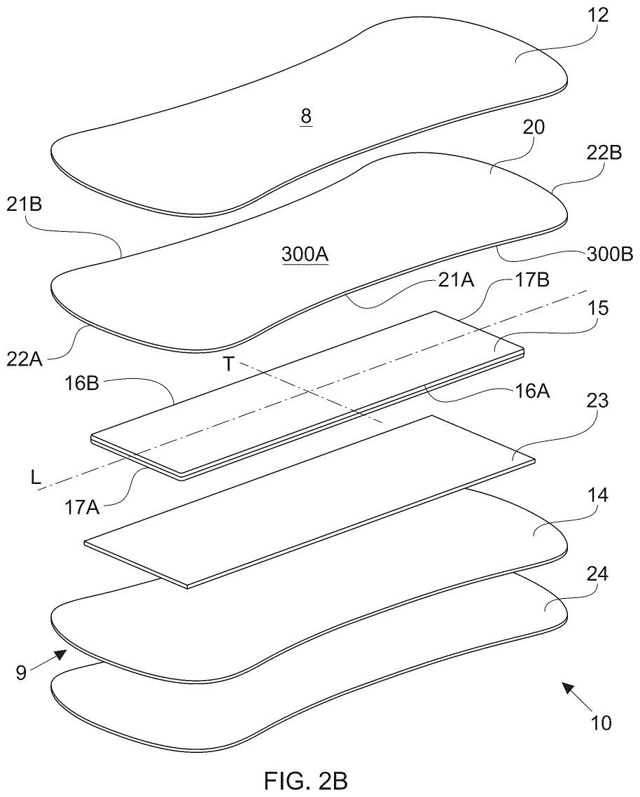

The absorbent article, also referred to herein as a liner, may include one or more layers. FIGS. 2A and 2B illustrate the different components or layers of an exemplary embodiment of an absorbent article. As illustrated in FIGS. 2A and 2B, the absorbent article includes a topsheet layer 12 forming at least a portion of the wearer-facing surface 8, a first barrier layer 23, a second barrier layer 14 forming at least a portion of the garment-facing side 9 of the absorbent article, and an absorbent core layer 15 situated between the topsheet layer 12 and second barrier layer 14. Additionally, in some embodiments, the absorbent article may also include a fluid management layer 20 positioned between the topsheet 12 and the absorbent core 15. As illustrated in FIG. 2B, the absorbent article may additionally include a release cover 24, which may be removed to expose an adhesive used to attach the absorbent article 10 to a user's undergarment.

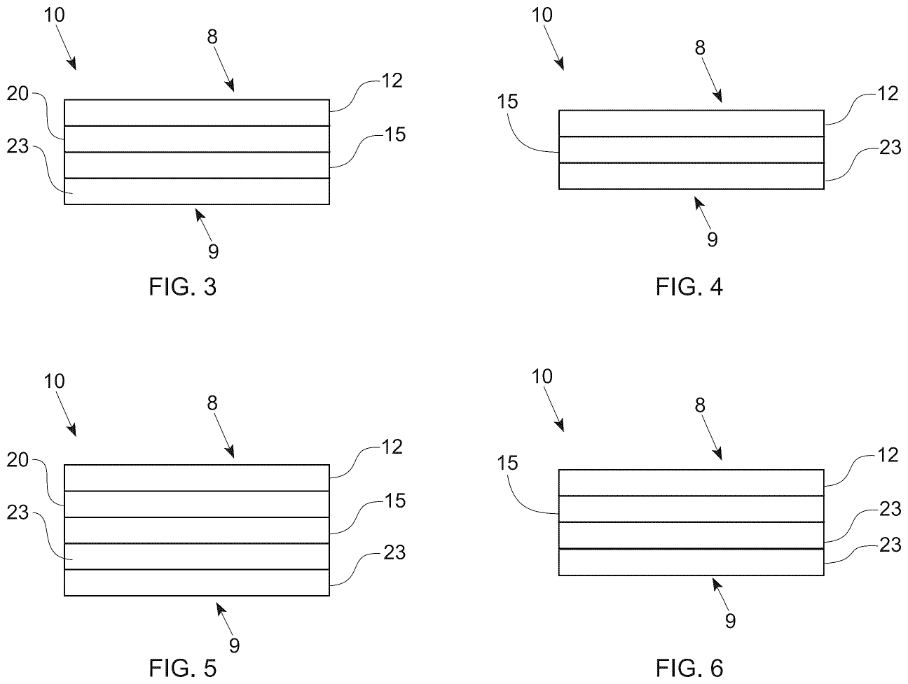

It is to be appreciated that the layers of the absorbent article 10 may be configured in various configurations. A cross sectional view of an absorbent article 10 including various layers is illustrated in FIGS. 3-6. It is to be appreciated that not every configuration is illustrated and other configurations are contemplated. As illustrated in FIG. 3, in some embodiments, the absorbent article 10 may include a topsheet layer 12, a fluid management layer 20, an absorbent core layer 15, and a first barrier layer 23. More specifically, the topsheet layer 12 may be disposed on the fluid management layer, and the fluid management layer may be disposed on the absorbent core, and the absorbent core may be disposed on the first barrier layer. As illustrated in FIG. 4, in some embodiments, the absorbent article 10 may include a topsheet layer 12, an absorbent core layer 15, and a first barrier layer 23. More specifically, the topsheet layer may be disposed on the absorbent core and the absorbent core may be disposed on the first barrier layer. As illustrated in FIG. 5, the absorbent article 10 may include a topsheet layer 12, a fluid management layer 20, an absorbent core 15, and two first barrier layers 23. More specifically, the topsheet layer may be disposed on the fluid management layer, and the fluid management layer may be disposed on the absorbent core layer, and the absorbent core layer may be disposed on two layers of the first barrier layer. As illustrated in FIG. 6, the absorbent article 10 may include a topsheet layer 12, an absorbent core 15, and two first barrier layers 23. It is to be appreciated that a release cover 24 may also be included in each of these embodiments although it is not illustrated. Additionally, as will be described in more detail herein, the first barrier layer and the second barrier layer may share or include properties of either the first barrier layer 23 or the second barrier layer 14. Thus, the embodiment as illustrated in FIGS. 3 and 4 may include either the first barrier layer 23 or the second barrier layer 14 or both of the first barrier layer 23 and the second barrier layer 14.

The topsheet layer and at least one of the first barrier layer 23 and the second barrier layer 14 may be joined together, at the perimeter edge of the absorbent article or in some other portion of the absorbent article 10, by any suitable mechanism, including but not limited to adhesive bonding, thermal/heat bonding, pressure bonding, ultrasonic bonding, dynamic mechanical bonding, a crimp seal, or by any other suitable bonding method, thereby retaining and holding the absorbent core layer 15 in a desired position. Additionally, the other layers as previously discussed may be joined together by one or more as mentioned herein.

As previously discussed, the absorbent article 10 of the present invention may be void of synthetic materials, such as fibers, films, and/or adhesives. “Synthetic” means that the polymerization did not happen in nature or was not naturally occurring. Synthetic materials are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Fibers that are not synthetic, for example, include cotton, flax, hemp, rayon, and jute. Substantially free of synthetic materials means that the article or component of the article is at least about 90% or at least 95% or at least about 98% by weight void of materials where the polymerization did not happen in nature or was not naturally occurring.

The absorbent article may include odor neutralizing materials, skin conditional materials, such as lotion, and/or perfumes so that the article carries a certain scent.

The absorbent article may be manufactured by mean generally known in the art, unless specifically address herein. One such manufacturing process is discussed in U.S. Pat. Publication Nos. 2020/0093656 and 2020/0093650, which are each incorporated by reference herein.

These and other features are described in further detail below.

### Topsheet

Known topsheets for feminine care hygiene products typically include a polymer film, such as hydroformed film, in combination with a polymer nonwoven substrate. However, the inventors have surprisingly found that a topsheet may be void of synthetic materials or substantially free of synthetic materials, such as synthetic fibers and films, and having the characteristics described herein, particularly in combination with the fluid management layer described below, that effectively prevents rewet and has sufficient acquisition time, as will be described in detail with respect to the examples and combinations herein. For example, a topsheet comprising fibers that comprise, consisting essentially of, or consist of cellulosic fibers has a rewet of less than about 10 g and an acquisition time of less than about 10 s, according to EDANA NWSP 80.10 (09) standard test method.

The article comprises a topsheet layer 12 which is liquid pervious. The topsheet layer may include a nonwoven web. The nonwoven web may include one strata of fibers or may be laminate of multiple nonwoven strata, which may include the same or different compositions of fibers. In some embodiments, the topsheet layer 12 includes a mix of hydrophobic and hydrophilic fibers. In some nonlimiting examples, the nonwoven web includes at least about 50%, or at least about 60% hydrophilic fibers by weight of the fibers. Additionally, or alternatively, the nonwoven may include at least about 30%, or at least about 40%, or at least about 50% of hydrophobic fibers by weight of the fibers. The nonwoven may include a majority of hydrophilic fibers and a minority of hydrophobic fibers. For instance, the nonwoven may include about 60% hydrophilic fibers and about 40% hydrophobic fibers. Fibers may be a size of about 0.9 to about 4.1 denier, or from about 1.2 to about 3.5 denier, or from about 1.3 to about 3 denier, or about 2 denier, reciting for said ranges every 0.1 increment therein. The topsheet layer may comprise at least about 50% or at least about 75% or at least about 90% of fibers having a decitex of from about 0.9 to about 4.1, according to the Fiber Decitex test method disclosed herein.

The topsheet layer 12 may be void of synthetic fibers and films or substantially free of synthetic fibers and films. The topsheet layer 12 may include a nonwoven web material that includes or consists predominately (by weight) or entirely of cellulose fibers. Cellulose fibers may be preferred to appeal to consumer preferences for natural products. Cellulosic fibers include, for example, fibers of cotton, flax, hemp, jute, kenaf or mixtures thereof. These fibers may be either naturally hydrophobic or suitably processed so as be rendered hydrophobic (or have increased hydrophobicity) and processed to be suitably soft. For example, unprocessed cotton fibers are naturally hydrophobic due to the natural wax-like substance that forms on the cotton fibers during growth. Cotton fibers that are mechanically cleaned retrain some of the natural hydrophobicity. Generally, cotton fibers that are chemically cleaned have reduced hydrophobicity given the chemical cleaning process removes or substantially removes the natural wax-like substance from the cotton fibers. There is a range of chemical cleaning processing that can be used to clean the cotton fibers and the resulting hydrophobicity of those fibers after undergoing the cleaning process is variable. Generally, the more thorough the cleaning process, the lower the resulting hydrophobicity. It is also to be appreciated that a combination of mechanical and chemical cleaning processes may be used. Further, subsequent processes may be used to add hydrophobicity or hydrophilicity to the cotton fibers. In other examples, fibers derived from cellulosic material, such as rayon (including viscose, lyocell, MODAL (a product of Lenzing AG, Lenzing, Austria) and cuprammonium rayon) may be used. For example, the topsheet layer may include at least one of rayon fibers and cotton fibers. The topsheet layer 12 may include at least one of hydrophobic viscose and hydrophilic viscose.

The topsheet layer may be apertured. Apertures are specifically useful for the topsheet layers comprising hydrophobic fibers. The apertures allow for fast liquid acquisition by allowing fluid to pass through the apertures to the subsequent layer. Although presence of the apertures might improve performance of the topsheet, namely speed of acquisition and rewet, apertures can as well have a merely aesthetic function and not contribute to the performance of the topsheet layer. Apertures may comprise a size of about 0.5 mm² to about 2.0 mm², reciting for said range every 0.1 mm² therein. Apertures might be predominantly round, or elliptic. Apertures might be regularly placed forming the mesh-like pattern or be placed in a way to create visually pleasing pattern. Usage of the apertured topsheet made of hydrophobic fibers might be specifically beneficial for delivering fast liquid acquisition and low rewet on the finished product, as will be demonstrated in the examples.

In some embodiments, the nonwoven web may be formed via a spunlaid process. In some embodiments, the nonwoven web material may be formed via a carding process. The fibers can then be integrated by various methods. Some exemplary processes for integrating topsheet fibers of a nonwoven web include spunlacing, needlepunching or chemical bonding with binders that a void of synthetic materials or substantially free of synthetic materials. Spunlacing uses a plurality of high pressure water jets to entangle fibers. Needlepunching involves the use of needles to push and/or pull fibers to entangle them with other fibers in the nonwoven.

The topsheet layer including cellulosic material may have a basis weight of at least about 23 gsm, or at least about 25 gsm, or up to about 60 gsm, or up to about 50 gsm, or from about 25 gsm to about 55 gsm, reciting for said range every 1 gsm increment therein. The basis weight may be calculated according to the Basis Weight test method herein.

Ideally to satisfy the consumer's desire to have an absorbent article that is perceived to be relatively more environmentally friendly, the topsheet layer 12 may consist of natural fibers, such as cellulosic fibers. However, the additional layers, such as the fluid management layer and the barrier layer, may be combined with a topsheet including non-cellulosic fibers or fibers that are not substantially free of or void of synthetic material. Again, this may not be ideal to the consumer but may provide a stepwise perceived improvement when combined with other layer(s) that are substantially free of or void of synthetic fibers and films. For example, the topsheet layer 12 may include fibers may be formed from polymeric materials, such as polyethylene (PE) and/or polyethylene terephthalate (PET). Fibers may be in the form of bi-component fibers. In nonlimiting examples, bicomponent fibers which may comprise PET as a core in combination with another polymer as a sheath. In further nonlimiting examples, PE may be used as a sheath in combination with a PET core. Suitable fibers may be staple fibers having a length of at least about 30 mm, or at least about 40 mm, or about at least about 50 mm, or up to about 55 mm, or from about 30 to about 55 mm, or from about 35 to about 52 mm, reciting for said range every 1 mm increment therein. In nonlimiting examples, staple fibers may have a length of about 38 mm.

### Fluid Management Layer

The fluid management layer 20 is disposed between the absorbent core layer 15 and the topsheet layer 12. Returning to FIG. 2, the fluid management layer 20 includes opposing end edges 22A and 22B which may extend generally parallel to a transverse axis T and may be straight or curved, and the fluid management layer 20 includes side edges 21A and 21B which may extend generally parallel to the longitudinal axis L and may be straight or curved. The fluid management layer of the present disclosure may be void of synthetic materials, such as synthetic fibers and films. The fluid management layer may be the same size as the absorbent core layer or smaller than the absorbent core layer in one or more dimensions. The fluid management layer may be the same size as the topsheet layer or smaller than the topsheet layer in one or more dimensions. The fluid management layer may be the same size as the absorbent core or a different size than the absorbent core layer in or more dimensions.

In addition to the softness and resiliency benefits, fluid acquisition and low rewet are additional benefits of absorbent articles of the present invention. Regarding fluid acquisition time, this attribute is key in making the user feel dry and clean and to prevent leaks. When the absorbent article takes a long time to drain liquid insults from the topsheet, users can feel wet. Additionally, when fluid stays on the topsheet for an extended period of time, users can feel like their skin is unclean. Further, in contrast to some menstrual pads, liners are often worn for long periods of time; and thus, there is a risk that compression during wear will draw fluids back to the surface at some point. In which case, the wearer will feel wet and sense failure in the product. The absorbent article of the present invention mitigates rewet due to its structure to relatively quickly desorb fluid from upper layers to the core and resiliency to prevent fluids from resurfacing upon being wet.

The fluid management layer provides increased caliper to the absorbent article which can translate into a softer feeling article. Additionally, the fluid management layer of the present disclosure can provide increased resiliency to the absorbent article. Typically, there is a tradeoff with resiliency and softness. Softer materials may have difficulty recovering their shape from insults of force in one or more directions, and the converse may be true for resilient materials. In the absorbent article context, resilient materials typically exhibit good recovery from insults of force; however, they are typically not perceived as being very soft. It is also worth noting that many absorbent articles can exhibit good resilience properties when dry; however, upon absorption of a liquid insult, their resiliency decreases substantially. The absorbent articles of the present disclosure exhibit good resiliency properties both in dry and wet conditions.

As noted previously, the fluid management layer is substantially free of or void of synthetic materials, such as fibers, films, and/or adhesives. The fluid management layer includes a nonwoven web material that includes or consists predominately (by weight) or entirely of cellulosic fibers. The cellulosic fibers may include, for example, fibers of cotton, flax, hemp, jute, kenaf, rayon, or mixtures thereof. The fluid management layer can provide capillary suction to “pull” fluid through the topsheet layer. The fluid management layer also can contain a gush by providing distribution functions to efficiently utilize the absorbent core, as well as provide intermediate storage until the absorbent core can accept fluid. In some embodiments, the fluid management layer may comprise an integrated, carded, nonwoven material. The fluid management layer of the present disclosure may comprise one or more carded webs, which may be subsequently integrated with one another. Each carded nonwoven web forms a stratum in the fluid management layer. The fibers of the fluid management layer may be staple fibers. In some embodiments, the fluid management layer may comprise an airlaid material, such as one or more airlaid layers of pulp fibers.

Absorbent articles in accordance with the present disclosure exhibit a soft cushiony feel, good resiliency, and fluid handling characteristics. The caliper of the fluid management layer therein is important. The fluid management layer has a caliper of from about 0.3 mm to about 1.6 mm or from about 0.5 to 1.6 mm, including all values within these ranges and any ranges created thereby. The caliper, also referred to herein as thickness, may be determined by the Caliper test method disclosed herein.

The fluid management layer may have a basis weight of from about 35 gsm to about 80 gsm, or from about 45 gsm to about 75 gsm, or from about 55 gsm to about 65 gsm, or from about 45 gsm to about 60 gsm, specifically including all values within these ranges and any ranges created thereby, according to the Basis Weight test method disclosed herein. In one specific example, the fluid management layer of the present disclosure can have a basis weight of between about 45 gsm to about 55 gsm. The basis weights of the fluid management layers of the present disclosure may be determined by the Basis Weight test method disclosed herein.

As will be discussed in additional detail hereafter, the types of fibers in the fluid management layer must be able to absorb fluid and provide resiliency so that the layer does not collapse upon absorption of fluid. It has been found that a fluid management layer comprising cellulosic fibers and/or fibers that are substantially free of or void of synthetic materials, may achieve these properties through a first embodiment including a carded layer including staple fibers and a second embodiment including an airlaid layer including pulp fibers.

In some embodiments, the fluid management layer may be an integrated, carded, nonwoven material. The fluid management layer may comprise one or more carded webs, which may be subsequently integrated with one another. Each carded nonwoven web forms a stratum in the fluid management layer. The fibers of the fluid management layer may be integrated through a spunlace process or a needlepunching process.

The fluid management layer may be formed from staple fibers. The fluid management layer may include cellulosic fibers. The cellulosic fibers may include at least one of cotton, flax, hemp, jute, kenaf, rayon, or mixtures thereof. The rayon fibers may include viscose. The cellulosic fibers may be at least one of hydrophobic and hydrophilic. For example, the fluid management layer may include at least one of hydrophobic and hydrophilic viscose fibers. The fluid management layer may include at least one of hydrophobic and hydrophilic cotton fibers. By including fibers that are both hydrophobic and hydrophilic, the hydrophobic fibers provide strength and resiliency to the layer by not absorbing fluid and thus maintaining their integrity when encountered by fluid, and the hydrophilic fibers allow the layer to absorb the fluid which pulls the fluid into the layer and away from the topsheet layer, which allows the absorbent article to control the fluid and feel relatively dry against the skin of the user.

However, it has surprisingly been found that the fluid management layer may include all hydrophilic fibers and still maintain the necessary resiliency to have sufficient rewet and acquisition time. Each fiber of the fluid management layer has an area moment of inertia. The area moment of inertia may be selected to achieve a desired level of rewet and acquisition time, which is influenced by the void volume created between the individual fibers. The area moment of inertia is related to the shape and size of the individual fiber which dictates the amount of void volume present in the layer.

For the comparative materials with synthetic fibers, void volume can be created in a layer using synthetic fibers, such as polyethylene and polypropylene, by adding heat and melting the fibers, or a portion thereof, so that a portion of the fibers bond to one another. This melting and bonding of fibers create a network of voids within the layer. However, for a fluid management layer that is substantially free of or void of synthetic fibers, the fibers cannot be melted and bonded because non-synthetic fiber, such as cellulosic fibers do not melt. Thus, to create the void volume necessary to achieve a desired rewet and acquisition time, the fiber shape and size needs to be optimized. This optimization can be achieved through the area moment of inertia.

It is worth noting that for a set basis weight of a fiber, larger diameter fibers can provide more void volume between adjacent fibers as compared to their smaller diameter counterparts. As such, the fiber size of the fibers in the fluid management layer can be important. For example, for a set percentage weight of fiber, as fiber size goes up there are fewer fibers per gram, and fewer fibers can equal more space between the fibers. Ideally, the fluid management layer should have void volume as well as some degree of capillarity to drain the topsheet.

With the above in mind, the inventors have also surprisingly discovered that careful selection of the fiber types in each of the strata in the fluid management layer and the linear densities of the fiber types can achieve the desired outcome of quick acquisition time and low rewet. For example, an absorbent article including the fluid management layer discussed herein may have an acquisition time of less than about 0.4 seconds and a rewet of less than about 0.10 g, according to the liquid Acquisition Time test method and the Standard rewet test method, each disclosed herein. The fiber types of the individual strata are discussed in additional detail hereafter.

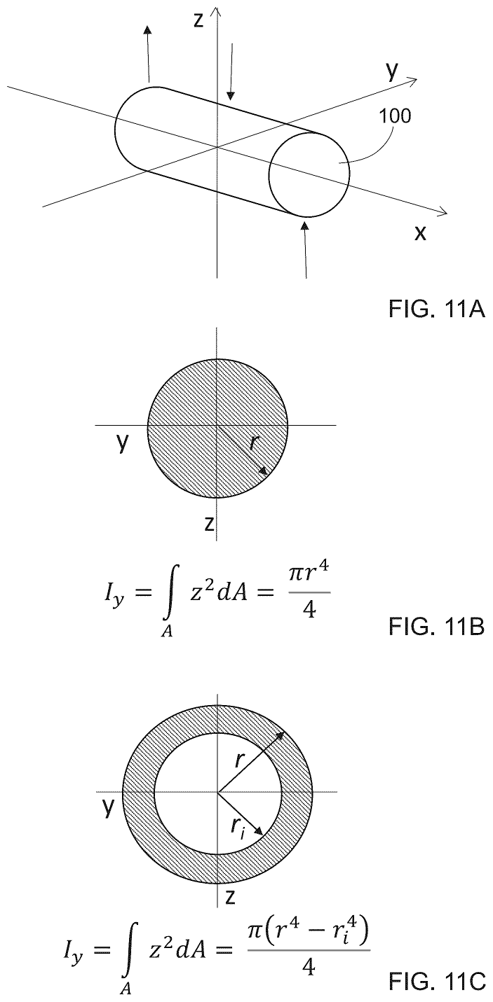

Area moment of inertia is defined by the fibers cross-sectional shape and fiber size. Area moment of inertia depends on the defined bending axis, which for this specific case is perpendicular to axis coming through the center of the fiber, as illustrated in FIG. 11A. As illustrated in FIG. 11A, the x-axis is coming through the center of the fiber 100, and the fiber 100 is bent around the y-axis. The area moment of inertia about the y-axis is calculated as:

I_{y}=∫z²dA
where z is the distance from the axis and the quantity I_{y} is the integral of squared distance (22) over the area A of the fiber cross-section. Therefore, when the large portion of the cross-sectional area of a given fiber is located further away from the centroid of the single fiber cross-section, this will lead to a higher area moment of inertia. Thus, fibers with the trilobal, hollow or other non-round cross-sectional area generally have a relatively higher area moment of inertia and, thus, are preferred. FIGS. 11B and 11C includes examples of fibers have a circular cross-sectional shape and their associated calculations for the area moment of inertia. As previously mentioned, the fibers of the fluid management layer may have a non-circular cross-sectional shape.

Table 1 below includes example calculations of various fiber cross-sectional shapes and their associated decitex (dTex). Cross sectional area of the fiber and fiber diameter can be measured via a suitable microscopy technique such as scanning electron microscopy (SEM, as disclosed in the test methods section under Fiber Decitex).

TABLE 1

| | Fiber diameter, | AMOI calculation | AMOI, |
|---|---|---|---|
| | μm | formula | μm⁴ |
| 1.7 dTex, solid round viscose fiber | 11.9 | $\frac{π {r}^{4}}{4}$ | 995 |
| 6.7 dTex, solid round viscose fiber | 23.7 | $\frac{π {r}^{4}}{4}$ | 15461 |
| 1.7 dTex, trilobal | Depends on the | I_{y} = ∫ z²dA | 2000-4000 |
| viscose fiber | cross-sectional | Integral calculated | |
| | shape, 13-25 | according | |
| | | to the shape | |
| 1.7 dTex, hollow round viscose fiber | For this example: Outer diameter: 16.7 Inner diameter: 11.7 | $\frac{π ( {r}^{4} - {r}_{i}^{4} )}{4}$ | 2906 |

Some suitable linear density values of absorbent fibers for use in the fluid management layers of the present disclosure are provided. For example, the linear density may range from about 1 dtex to about 10 dtex, or from about 1.4 dtex to about 8 dtex, or from about 2 dtex to about 6 dtex, specifically reciting all values within these ranges and any ranges created thereby. In one specific example, the absorbent fiber may comprise a linear density of about 1.7 dtex. The dtex of the fibers may be determined via the Fiber Decitex test method disclosed herein. The fluid management layer may comprise greater than about 50% of staple fibers having a decitex of from about 1.3 to about 10, or greater than about 75% of the staple fibers having a decitex of from about 1.3 to about 10, or greater than about 90% of the staple fibers having a decitex of from about 1.3 to about 10.

The fibers of the fluid management layer may have any suitable shape. Some examples include trilobal, “H,” “Y,” “X,” “T,” round, or flat ribbon. Further, the fibers can be solid, hollow or multi-hollow. The trilobal or other multi-lobed shape can improve wicking and improve masking. Suitable trilobal rayon is available from Kelheim Fibres and sold under the trade name Galaxy. Each stratum may comprise a different shape of fiber.

Additionally, to achieve the desired product performance as previously discussed, it has been surprisingly found that the cross-sectional shape of the individual fibers can be selected to optimize these performance characteristics, such as rewet and acquisition time. More specifically, it has been found that the fluid management layer is optimized when the individual fibers have a decitex of from about 1.3 to about 10 or from about 5 to about 7, and when the fibers have an area moment of inertia of from about 2,000 μm⁴ to 95,000 μm⁴ or of from about 3,749 μm⁴ to 85,000 μm⁴ or from about 5,000 μm⁴ to 75,000 μm⁴ or from about 7,000 μm⁴ to 40,000 μm⁴ or about 7,000 μm⁴ to 25,000 μm⁴ or from about 7,000 μm⁴ to 15,000 μm⁴, calculated as previously discussed herein. As previously mentioned, by including fibers with the aforementioned area moment of inertia, the fluid management layer will have sufficient void volume and capillary effect to drawn fluid into the absorbent core and will exhibit an acquisition time of less than about 0.4 seconds and a rewet of less than about 0.10 g, according to the Liquid Acquisition Time test method and the Standard Rewet test method, each disclosed herein.

It is also to be appreciated that the fluid management layer may have different fibers. In some embodiments, a carded fluid management layer may include a first plurality of fibers having a first cross-sectional shape and a second plurality of fibers having a second cross-sectional shape, and the first cross-sectional shape and the second cross-sectional shape may be the same or different. The cross sectional shape of the fibers may be trilobal, circular, elliptical, or any other shape. The fibers may also be hollow and/or solid. The fluid management layer may include a first plurality of fibers that have a first decitex and a second plurality of fibers that have a second decitex, and the first decitex and the second decitex may be the same or different. For example, the first decitex may be at least 1.2 times greater than the second decitex. Further, fluid management layer may include a first plurality of fibers that have a first area moment of inertia and a second plurality of fibers that have a second area moment of inertia, and the first area moment of inertia and the second area moment of inertia may be the same or different. When the first plurality of the fibers has first area moment of inertia, and second plurality of the fibers has second area moment of inertia, the overall layer area moment of inertia is calculated as the weighted average of the area moment of inertia of the component fibers. The amount of the first plurality of fibers and the second plurality of fibers may be varied to achieve the desired performance of the fluid management layer. For example, the fluid management layer may include at least 40% of a first plurality of fibers and at least 60% of a second plurality of fibers. Although the properties of the individual fibers may be varied, each of the fibers of the fluid management layer has a decitex and moment of inertia within the recited aforementioned ranges to achieve the desired performance, such as rewet and acquisition time. It is also to be appreciated a third plurality and a fourth plurality of fibers also may be included in the fluid management layer.

To enhance the stabilizing effect of the fluid management layer, crimped, carded fibers may be utilized. For example, these fibers may be mechanically crimped and/or may have a chemically induced crimp due to the variable skin thickness formed during creation of the fibers.

As noted previously, the amount of fibers that absorb fluid, hydrophilic fibers, can impact the absorption of liquid insults to the wearer-facing surface or topsheet. However, when absorbent fibers absorb liquid, they tend to lose some of their structural integrity. If the desired resilience cannot be maintained with 100% hydrophilic fibers, hydrophobic fibers may be incorporated into the fluid management layer. Examples of resilient and strong fibers are those hydrophobic fibers, such as hydrophobic viscose and hydrophobic cotton. The fluid management layer of the present disclosure may comprise from about 10 percent to about 70 percent, or from about 15 percent to about 60 percent, or from about 25 percent to about 50 percent by weight of fibers that are hydrophobic, specifically reciting all values within these ranges and any ranges created thereby. In one specific example, the fluid management layer may comprise about 15 percent by weight fibers that are hydrophobic. In another specific example, the fluid management layer may comprise about 20 percent by weight of fibers that are hydrophobic. Generally, hydrophobic fibers do not absorb fluid and, thus, it is believed that these fibers better maintain their shape and integrity. As previously discussed, although hydrophobic fibers need not be present in the fluid management layer, these fibers may be added to increase the resilience and performance properties of the fluid management layer. A ratio of hydrophobic fibers to hydrophilic fibers in the fluid management layers can be from about 1:7 to about 2:1, or from about 1:4 to about 1.5:1, or from about 1:2 to about 1:1, specifically reciting all values within these ranges and any ranges created thereby.

Any suitable material for the fibers that absorb fluid may be utilized. Some examples of fibers include cotton, pulp, rayon or regenerated cellulose or combinations thereof. In one example, the fluid management layer 20 may comprise viscose fibers. The length of the fibers can be in the range of about 20 mm to about 100 mm, or more preferably about 30 mm to about 50 mm or most preferably about 35 mm to about 45 mm, specifically reciting all values within these ranges and any ranges created thereby. In general, the fiber length of pulp is from about 4 to 6 mm and cannot be processed in conventional carding machines because the pulp fibers are too short. So, if pulp is desired as a fiber in the fluid management layer, then additional processing to add pulp to the carded webs may be required. As an example, pulp may be airlaid between carded webs with the combination being subsequently integrated.

A schematic representation of an exemplary fluid management layer in accordance with the present disclosure is provided in FIGS. 2A and 2B. As shown, the fluid management layer 20 comprises the first surface 300A and the opposing second surface 300B. Between the first surface 300A and the second surface 300B, the fluid management layer 20 may include two or more strata along the Z-direction. The fluid management layer may be formed of one or more nonwoven webs. The nonwoven web may be formed from a carding process, as is known in the art, and, in some embodiments, an integration process, such as needle-punching and spunlace, as is also well known in the art.

In another embodiment, the fluid management layer includes a plurality of pulp fibers that have undergone an airlaid process. The pulp fibers are laid down in an airlaid forming process. The pulp fibers are then compressed forming hydrogen bonds between a portion of the plurality of pulp fibers. A carrier tissue layer comprising a cellulosic film might be used to support and/or cover fibers during the airlaying process. It is to be appreciated that a binder that is free of synthetic polymers may be used to bond the portion of the plurality of pulp fibers to form the fluid management layer. Pulp fibers may be at least one of softwood pulp, hardwood pulp, bamboo fibers, hemp fibers, and eucalyptus fibers. In addition to conventional pulp fibers cellulose wadding, fluffed cellulose fibers, rayon fibers, and textile fibers can be used. The airlaid fluid management layer may have a basis weight of from about 45 gsm to about 75 gsm or from about 60 gsm to about 80 gsm, according to the Basis Weight test method disclosed herein, and a caliper of from about 0.3 mm to about 0.9 mm, according to the Caliper test method disclosed herein. The fibers of the airlaid fluid management layer may have a fiber length of from about 0.5 mm to about 8 mm or from about 1.5 mm to about 5 mm or from about 1.5 mm to about 3 mm. The fibers may include at least one of hydrophilic and hydrophobic pulp fibers. The pulp fibers may be naturally hydrophobic or may be treated to be hydrophobic. A fluid management layer may include one or more stratum including an airlaid layer.

In summary, the fluid management layer may include be a spunlace layer having a basis weight of from about 45 gsm to about 75 gsm, according to the basis weight test method disclosed herein, a caliper of from about 0.5 mm to about 1.3 mm, according to the Caliper test method disclosed herein, and a decitex of from about 0.5 to about 10 or from about 1.3 to about 10 or from about 1.5 to about 10, according to the Decitex test method disclosed herein. The fluid management layer may include be an airlaid layer having a basis weight of from about 60 gsm to about 80 gsm, according to the basis weight test method disclosed herein, a caliper of from about 0.3 mm to about 1 mm, according to the Caliper test method disclosed herein, and a decitex of from about 1.3 to about 3.5, according to the Decitex test method disclosed herein. The fluid management layer may include be a needlepunched layer having a basis weight of from about 45 gsm to about 75 gsm, according to the basis weight test method disclosed herein, a caliper of from about 0.5 mm to about 1.6 mm, according to the Caliper test method disclosed herein, and a decitex of from about 1.5 to about 10, according to the Decitex test method disclosed herein. The fluid management layer may include be a carded staple fiber having a basis weight of from about 45 gsm to about 75 gsm, according to the basis weight test method disclosed herein, a caliper of from about 0.5 mm to about 1.6 mm, according to the Caliper test method disclosed herein, and a decitex of from about 1.5 to about 10, according to the Decitex test method disclosed herein.

### Absorbent Core

The article further comprises an absorbent core layer 15, which serves as a storage layer for bodily exudates. The configuration and construction of the absorbent core 15 may vary (e.g., the absorbent core 15 may have varying caliper zones, a hydrophilic gradient, a superabsorbent gradient, or lower average density and lower average basis weight acquisition zones). The absorbent core 15 include opposing end edges 17A and 17B which may extend generally parallel to the transverse axis T and may be straight or curved. The absorbent core 15 may include side edges 16A and 16B which extend generally parallel to the longitudinal axis L and may be straight or curved. The size and absorbent capacity of the absorbent core 15 may also be varied to accommodate a variety of wearers. However, the total absorbent capacity of the absorbent core 15 should be compatible with the design loading and the intended use of the disposable absorbent article. For example, the absorbent capacity of the absorbent core layer may be selected in view of the other adjacent layers in the absorbent article, such as the fluid management layer and/or the barrier layer(s).

The absorbent core can contain conventional absorbent materials. The absorbent core may include fibers that includes or consists predominately (by weight) or entirely of cellulosic fibers. In addition to conventional absorbent materials such as creped cellulose wadding, fluffed cellulose fibers, rayon fibers, wood pulp fibers also known as airfelt or pulp fibers, and textile fibers, the core often includes superabsorbent material that imbibes fluids and form hydrogels. Such materials are also known as absorbent gelling materials (AGM) and may be included in particle form. AGM is typically capable of absorbing large quantities of body fluids and retaining them under moderate pressures. In some embodiments, the absorbent core layer may be substantially free of or void of synthetic fiber and films. For those absorbent articles that are substantially free of or void of synthetic materials, these absorbent articles would be void of superabsorbent material as this material is synthetic. However, as previously discussed, consumers desire a range of products and, thus, an absorbent core layer comprising superabsorbent material may be combined with other layers, such as the topsheet layer, the barrier layer(s), and/or the fluid management layers that may be substantially free of or void of synthetic materials or void of synthetic fibers and films.

In some embodiments, the absorbent core is an airlaid absorbent core including pulp fibers, or pulp fibers and absorbent gelling material. More specifically, the absorbent core layer may include a stratum of cellulosic material, such as cellulosic fibers. The cellulosic fibers may be pulp fibers. The pulp fibers may be combined into a stratum via airlaid technology. Pulp fibers may be at least one of softwood pulp, hardwood pulp, bamboo fibers, hemp fibers, and eucalyptus fibers. In some embodiments, the cellulosic fibers may be stapled and crimped fibers, which may include rayon fiber that are combined via a spunlace or needlepunch process. The absorbent core layer may include a single layer or two or more stratum of cellulosic material. For example, the absorbent core may include at least one stratum with pulp fibers and at least one stratum with rayon fibers.

The absorbent core may have a basis weight of at least about 75 gsm, or at least about 100 gsm, or at least about 140 gsm, or at least about 144 gsm, or at least about 150 gsm, or at least about 160 gsm, or at least about 165 gsm, or at least about 170 gsm, or from about 75 gsm to about 230 gsm or from about 90 gsm to about 185 gsm, reciting for said range every 5 gsm increment therein, according to the Basis Weight test method herein. The absorbent core layer may have a caliper of from about 0.35 mm to about 5 mm, according to the Caliper test method disclosed herein.

The absorbent core layer 15 of the present disclosure may comprise any suitable shape including but not limited to an oval, a disco-rectangle, a rectangle, an asymmetric shape, and an hourglass. For example, in some forms of the present invention, the absorbent core 205 may comprise a contoured shape, e.g., narrower in the intermediate region than in the end regions. As yet another example, the absorbent core may comprise a tapered shape having a wider portion in one end region of the pad which tapers to a narrower end region in the other end region of the pad. The absorbent core 15 may comprise varying stiffness in the MD and CD.

The capacity and properties of the absorbent core layer 15 may be selected in view of at least one of the fluid management layer 20 and the first barrier layer 23 and/or the second barrier layer 14. For example, for a fluid management layer 20 that retains fluid for a longer period of time, the absorbent core may have a relatively lower capacity. For example, the absorbent core layer may have a total capacity of below about 5.7 g/liner, and still deliver a rewet of less than about 0.02 g, according to the Standard Rewet test method, and a finished product acquisition time of less than about 1 second, according to the Liquid Acquisition Time test method disclosed herein. For an absorbent core layer 15 that has a relatively greater absorbent capacity than is necessary for the intended use of the absorbent article, the fluid management layer 20 may have a relatively lower performance. For example, because the possibility of rewet is diminished due to the relatively higher capacity of the absorbent core layer, the fluid management layer may have a relatively lower basis weight, lower area moment of inertia fibers and/or include fewer absorbent fibers.

Additionally, or alternatively, the first barrier layer and/or second barrier layer properties may be selected in view of the absorbent core layer. For example, for an absorbent core layer 15 that has an absorbent capacity the exceeds the capacity of the absorbent article for normal, intended use, the first barrier layer may need not be as resistant to fluid because it would be less likely that fluid would reach the first barrier layer given the capacity of the core. Thus, the first barrier layer may have a relatively larger pore size allowing for greater breathability and reduced fluid barrier properties. The absorbent core layer 15 may be selected such that to reduce cost, the absorbent capacity was design to accept the normal fluid for the intended use or a slightly reduced amount of fluid than the intended use. Thus, fluid may be relatively more likely to not be retained by the absorbent core and the first barrier layer may have minimal or no pores to provide a barrier between the absorbent core layer and the second barrier layer and/or the undergarment of the user.

These details will be discussed in greater detail with respect to the examples provided herein.

### First Barrier Layer

The absorbent article 10 may include a first barrier layer 23 as is illustrated in FIGS. 2 and 2B. The first barrier layer 23 may be disposed adjacent to a surface of the absorbent core layer 15. The first barrier layer 23 may be configured to prevent or substantially prevent any fluid that passes through or around the absorbent core layer 15 from leaking onto the user's garments. The first barrier layer 23 may include, consist essentially of, or consist of at least one of cellulosic fibers and cellulosic film. For example, the first barrier layer 23 may include Kraft paper, creped paper, parchment paper, cellophane, and/or regenerated cellulosic film. The first barrier layer 23 is preferably durable, discrete, and comfortable. More specifically, the materials of the first barrier layer 23 are desired to be flexible to allow for conformability to the user, strong to withstand use without tearing or breaking down due to fluid insult, and relatively low noise producing so that movement does not cause unwanted attention. Further, as previously discussed, it is preferred that due to the regular use of liners that these absorbent articles include materials that relatively reduced adverse impact on the environment. Thus, the first barrier layer 23 may be substantially free of or void of synthetic fiber and films.

The first barrier layer 23 may be impervious, or substantially impervious, to fluid (e.g., vaginal discharge, urine, menstrual fluid) and may be manufactured from a substrate or web. As used herein, the term “flexible” refers to materials which are compliant and will readily conform to the general shape and contours of the human body. The first barrier layer 23 may prevent, or at least substantially inhibit, fluids absorbed and contained within the absorbent core layer 15 from escaping and reaching articles of the user's clothing, such as underpants and outer clothing. However, in some instances, the first barrier layer 23 may be made and/or adapted to permit vapor to escape from the absorbent core layer 15 (i.e., the first barrier layer 23 may be made to be breathable), while in other instances the first barrier layer 23 may be made so as not to permit vapors to escape (i.e., it may be made to be non-breathable). Traditionally, the barrier layers have been made from polymeric film such as thermoplastic films of polyethylene or polypropylene. For example, a known, suitable material for a barrier layer is a thermoplastic film having a thickness of from about 0.012 mm to about 0.051 mm, according to the Caliper test method disclosed herein. However, as previously discussed, it is desirable for users to have an absorbent article that is generally void of synthetic fibers and films and/or has a relatively reduced adverse impact on the environment. Thus, the first barrier layer 23 may be manufactured from one or more stratum of cellulosic materials and may be void of synthetic fibers and films.

The first barrier layer 23 may include one or more stratum including, consisting essentially of, or consisting of a plurality of cellulosic fibers. The cellulosic fibers may include pulp, hemp, cotton, rayon, or regenerated cellulose. In some embodiments, the first barrier layer 23 may comprise, consist essentially of, or consist of paper. The term “paper” refers to a material manufactured in sheets from pulp fibers, which is a collection of individual fibers. The pulp fibers may be wood pulp fibers or other fibrous substances and may include additives. The paper may be Kraft paper, creped paper, and creped Kraft paper. Paper making additives known in the art are fillers like calcium carbonate (from Chalk or ground limestone), Kaolin clays, titanium dioxide, talc, gypsum, Aluminum trihydroxide and silica products. Fillers may control the pore size of papers and improve opacity and brightness. In addition to fillers, additives can include sizing agents, strength agents, such as cationic starches, and defoamers. The paper may be made with pulp obtained by chemical processes like the Kraft process or Sulfite process. Paper made from kraft pulp is generally preferred for its strength and purity of fiber with low lignin content. All types of wood and non-wood species like bamboo and kenaf can be used in the kraft process. Alternatively, the paper pulp may be obtained by mechanical processes and/or chemical mechanical processes. For example, a paper may be made with a majority of softwood pulp fibers which are known for their strength and higher fiber length. One example of soft wood pulp fibers is made from Northern pine.

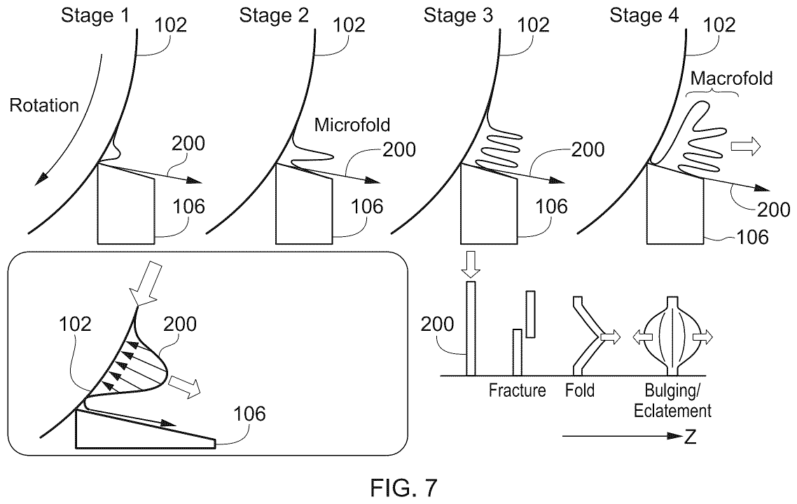

The term “Kraft paper” refers to a paper produced from wood pulp by the Kraft process. The Kraft process removes almost all lignin from the wood, which results in almost pure cellulose fibers. The Kraft process is known in the art. A Kraft paper sheet is characterized by overall good strength properties and relatively high porosity, such as for those Kraft papers that have a basis weight of less than about 50 gsm. The term “creped” or “creping” refers to a mechanical process for creating low density, increased caliper paper. Creping is a mechanical deformation process where paper is gathered in small folds. Creping may be done in-line with the paper making process after the paper web has been formed or as an off-line process where flat paper is the feedstock. The process involves positioning the paper web about the outer circumferential surface of a cylinder 102, which may be a Yankee cylinder that is used to dry the paper web, and engaging the paper web with a creping blade 106, as illustrated in FIG. 7. Impact of the paper web or sheet 200 against the crepe blade 106 generates large forces within the paper sheet 200, partially disrupting the inter-fiber hydrogen bonds, distorting the fibers and forming micro- and macro-folds in the paper web. The expanded and buckled portion of the web releases from the cylinder 102 surface for a short distance, as shown in Stage 1 of FIG. 7. Thus, a small ridge or crepe forms, before the held sheet re-impacts against the crepe blade 106, and the process repeats. Completed crepes are constantly moving away as the sheet 200 is wound up onto the parent roll 108. The crepe process shortens the length of the sheet 200 while increasing its caliper, thus the parent roll 108 rotates slower than the cylinder 102. The crepe itself is not uniform, consisting of large folds (macrofolds, as shown in Stage 4) interspersed with many smaller folds (microfolds, as shown in Stage 3). The creping process makes the crepe paper stretchable, at least in the direction perpendicular to the ridges formed by the creping process, and after being stretched, the crepe paper may remain somewhat clastic. Creped paper is generally more elastic and has more stretch and elongation than paper that has not been subjected to a creping process. Thus, stretchability and elasticity make crepe paper a suitable replacement for plastic layers in liners.

As used herein, the term “percent (%) crepe” is defined as the difference in speed between the Yankee dryer or creping process surface and the wind-up reel as a percentage of the Yankee or creping process surface speed in a creping process. In other words, percent crepe is the net percentage by which the traveling web is foreshortened relative to its length while on the Yankee dryer or creping process surface. A higher percent crepe generally may be associated with a higher amplitude on the creping ridge and/or a high frequency of crepe ridges per unit length in the MD direction of the creped substrate. A higher percent crepe substrate may also be associated with its higher stretch, higher elongation, higher elongation at failure, and higher MD stretch and elongation at failure properties. Generally, creped substrates may also have their internal bonding disrupted or debonded, so as also to contribute to the creped substrate's higher stretch, higher elongation, higher elongation at failure, and higher MD stretch and elongation at failure properties.

In some embodiments, the first barrier layer 23 or other barrier layer of the present invention may comprise, consist essentially of, or consist of creped paper. The creped paper may be made using a process wherein the % crepe is greater than or equal to about 0%, 5%, 10%, 15%, 20%, 25%, 30%, 35%, 40%. Alternatively, the % crepe may range from about 0 to about 50%, about 1% to about 40%, about 3% to about 30%, about 4% to about 25%, about 5% to about 20%, or about 6% to about 10%. In yet one other embodiment, the % crepe is substantially equal to 0% which means the crepe ridges induced in the creped substrate have been pulled out or substantially eliminated by the reeling speed being the same as the creping surface speed. When the first barrier layer 23 or other barrier layer of the present invention comprises, consist essentially of, or consist of creped paper with a percent crepe substantially equal to 0%, there may be substantially no visible sign of crepe ridges in such a material.

As previously discussed, the first fluid barrier layer needs to be strong enough to withstand the forces the user placed on the product during wear, removal, and initial placement and to withstand the insult of fluid. To obtain this strength, the fluid barrier layer 23 may include a blend of hardwood and softwood fibers, preferably a majority of softwood fibers, which may provide the toughness needed for surviving manufacturing process, such as the creping process, and for maintaining sufficient strength and bonding integrity within the fibers. The fluid barrier layer 23 may comprise about 50% by weight to about 80% by weight of softwood fibers. The fluid barrier layer 23 may comprise about 20% by weight to about 50% by weight of hardwood fibers. The hardwood fibers may be selected from the group consisting of maple, oak, elm, birch, poplar, aspen or eucalyptus fibers, and combinations thereof, preferably birch fibers. The softwood fibers may be selected from the group consisting of pines, firs, spruces, hemlocks, larch, and combinations thereof, preferably northern pine fibers. The fluid barrier layer 23 may contain other natural fibers from plants such as cotton, flax, bamboo, wheat straw, red algae and/or other seaweeds, and hemp. The fluid barrier layer 23 may also contain recycled fibers. The fibers used in the fluid barrier layer 23 may have a Canadian Standard Freeness of at least 200 mL, more specifically at least 300 mL, more specifically still at least 400 mL, and most specifically at least 500 mL. For example, the fluid barrier layer 23 may include paper having at least about 50% of Northern softwood fibers to provide the necessary strength for being creped and to withstand use in an absorbent article.

The fluid barrier layer 23 must maintain sufficient strength when insulted with fluids and when subject to prolonged use in a relatively moist environment. It has been found that the fluid barrier layer 23 maintains sufficient strength when the materials of the fluid layer have a dry burst strength of at least about 80 kPa or at least about 100 kPa or at least about 200 kPa, and/or wet burst strength of at least about 10 kPa or at least about 12 kPa at least about 14 kPa or at least about 24 kPa. The wet burst strength should be at least 10% of the dry burst strength for the barrier layer 23 to maintain sufficient strength. Materials that may be used for the fluid barrier layer 23 and achieve the dry burst strength and wet burst strength as previously specified include, papers typically used in filtering applications. For example, Filter Paper Grade 3w, 3h and 3hw from Sartorius made from pulp and cotton linters having a basis weight of 65 gsm has an acceptable wet burst strength value of 15 kPa. Crepe filter paper Grade FT55 made of pulp and cotton linters with a basis weight of 55 gsm has an acceptable wet burst strength of 20 kPa.

To achieve these values of the wet burst strength the barrier layer(s) may be hydrophobic to prevent fluid from being absorbed into the barrier layer, which is cellulosic. Cellulosic materials generally swell when in contact with fluid resulting in lowering the strength of the material. To prevent the barrier layer from swelling and reducing its strength, the barrier layer may be hydrophobic so that the layer repels the fluid and prevents the fluid from entering the pores while the fluid is absorbent by other layers in the absorbent article, such as the absorbent core layer. The hydrophobicity may be achieved through hydrophobic fibers that are used to form the barrier layer or coatings applied to the barrier layer, such as a wax coating, and/or heat and pressure treatment that alters the surface energy of the fibers in the layer. These coatings may include natural waxes such as beeswax, rice brown wax, candelilla wax, carnauba wax, soy wax, and other vegetable waxes. The coatings may include blends of these natural waxes, which may contain vegetable oils. Hydrophobic coatings can also be created from natural hydrophobic polymers, such as lignin, and proteins containing non-polar amino acids such as casein, collagen, silk fibroin, beta keratin, glycine rich proteins from rice, soy and other vegetable sources, seaweed proteins, zein protein, and soy protein. The hydrophobicity of the first barrier layer 23 may be greater than about 10 seconds or greater than about 12 seconds or greater than about 15 or greater than about 18 seconds, according to the Water Repellency test method disclosed herein. The coatings may be substantially free of or void of synthetic materials. The coating may be applied such that the coating has a basis weight of from about 0.5 gsm to about 1.0 gsm. The coating, such as wax, may be applied to at least one of the first surface and the second surface of the first barrier layer. The coating may be applied during the manufacture of the barrier layer using any method as is known in the art to achieve adequate hydrophobicity.

For embodiments wherein the first barrier layer 23 includes a porous material, such as paper, it has been found that to achieve the desired hydrophobicity of the paper, the porosity of the paper needs to be managed. It has been found that for a first barrier layer 23 including a porous material, the material should have a certain pore size so that fluid does not leak through. It has been found that the largest pore size, also referred to the bubble point pore size, should be less than 20 microns, less than 18 microns, less than 15 microns, or less than 13 microns, or less than 9 microns, or less than 7 microns, according to the Capillary Flow Porometry test method disclosed herein. The porosity of the material, such as paper, may be reduced through the use of coatings applied to one or more surfaces of the material. The aforementioned coatings may be used to decrease the porosity of the material. Additionally, a primer may be applied prior to the coating or may be applied as the coating. For example, to increase the efficacy of the coating and/or to minimize the amount of coating, a primer may be applied to one or more surfaces of a paper to reduce the porosity of the paper by reducing the size of the pores in the paper. Primers may also improve the appearance of printed graphics, maintaining the definition of lines and reducing dot gains. The preferred primers are water-based and/or water soluble. The primer may be formulated with polymer resins, such as nitrocellulose, polyamides, acrylic polymers, polyurethanes, and adhesion promoters, such as polyimides, silanes, and aziridine. The primer formulation may also comprise biobased polymers, such as cellulose, starch, chitin, chitosan, xylan, other types of hemicelluloses, and polyesters derived from vegetable oils. The primer formulation may also contain minerals, such as clays, silicas, talc, and calcium carbonate (sometimes referred to as inorganic fillers).

Additionally, the first barrier layer 23 may include a porous material that has a certain repellency. The repellency is the ability of the material to repel a fluid. The repellency is measured by determining how long it takes a fluid to penetrate from the upper surface to the lower surface of the material, and thus, is a measurement of the hydrophobicity of the material. Generally, for a material of the first barrier layer 23, the material having a larger pore size need to have a greater repellency and for materials having a smaller pore size the repellency may be less. For example, a first barrier layer 23 comprising a material with a pore size of greater than 5 microns, the material should have a repellency of at least 20 seconds. For a first barrier layer 23 comprising a material with a pore size of less than 5 microns, the material should have a repellency of at least 10 seconds. The pore size of the material is determined by the Capillary Flow Porometry test method disclosed herein.

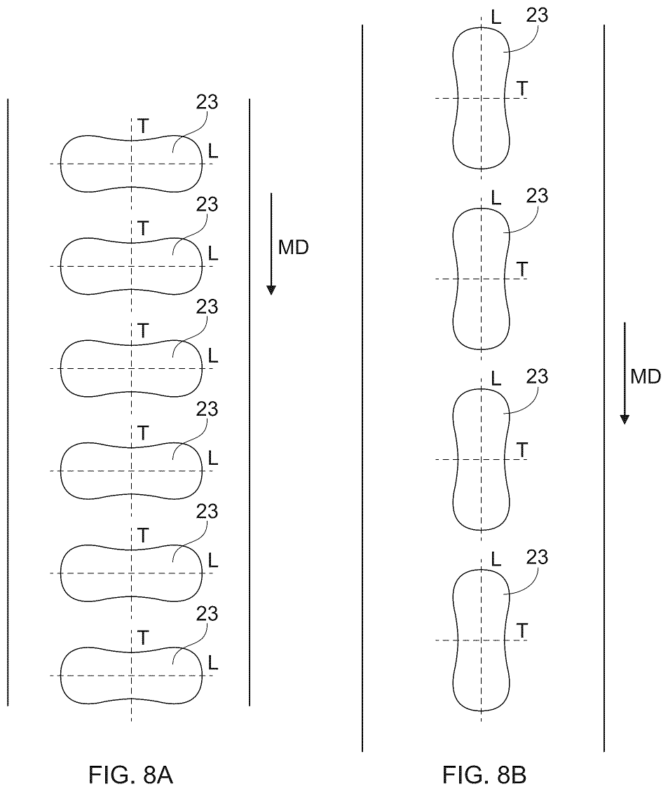

The first barrier layer 23 may be configured to stretch in a machine direction MD during manufacture such as illustrated in FIGS. 8A and 8B. In some embodiments, such as illustrated in FIG. 8A, the barrier layer may be manufactured such that the as the layer advances through the manufacturing process, the longitudinal axis of the barrier layer is substantially perpendicular to the machine direction MD. Thus, to allow the barrier layer to stretch and not break or bunch during the manufacturing process, the barrier layer 23 is configured to stretch in a direction, a stretch direction SD, substantially parallel to the machine direction MD. In some embodiments, such as illustrated in FIG. 8B, the barrier layer may be manufactured such that the as the layer advances through the manufacturing process, the longitudinal axis of the barrier layer is substantially parallel to the machine direction MD. Thus, to allow the barrier layer to stretch and not break or bunch during the manufacturing process, the barrier layer 23 is configured to stretch in a direction, a stretch direction SD, substantially parallel to the machine direction MD. The stretch direction of the barrier layer 23 may be dependent on the machine direction or the direction in which stretch is desired to prevent tearing or bunching of the layer.

The first barrier layer 23 may have an unstretched length, which is the length of the layer when no external force is applied to the layer, and a stretched length, which is the length of the layer when an external force is applied to the layer. The stretched length may be about 5%, or about 8%, or about 10%, or about 15%, or about 18%, or about 20% greater than the unstretched length, which may be measured according to standard test method ISO1924-3. In some embodiments, the first barrier layer 23 comprises paper and to provide stretch to the paper, the paper is creped. The first barrier layer 23 including creped paper has a plurality of ridges that extend in a ridge direction, and the first barrier layer is configured to stretch in a stretch direction that is substantially perpendicular to the ridge direction. The ridge direction may be substantially perpendicular to the machine direction, and/or the ridge direction may be substantially perpendicular to or parallel to the longitudinal axis L of the absorbent article 10. The each of the plurality of ridges may be continuous or discontinuous. The ability of the first barrier layer 23 to stretch in the stretch direction allows for the layer to be manufactured in a high-speed process without tearing or becoming damaged due to the tension placed on the layer during the high-speed processing. Further, the ability of the first barrier layer 23 to stretch prevents the layer from tearing or becoming damaged while being worn. Stated another way, the ability of the first barrier layer to stretch allows the layer to move with the user and better conform the user during movement. At least one of the first surface and the second surface of the first barrier layer may comprise a plurality of ridges or undulations that protrude from the planar surface of the layer.

Accordingly, in addition to providing stretch, the first barrier layer must have sufficient strength to endure the manufacturing process and regular use. For a first barrier layer comprising paper, the first barrier layer 23 may have a machine direction web modulus in the range of 1%-2% strain of less than about 600 N/cm, or less than about 550 N/cm, or less than about 450 N/cm, according to the Web Modulus test method disclosed herein and specifically reciting all values within these ranges and any ranges created thereby. It has been found that a layer having a machine direction web modulus of greater than 600 N/cm results in issues with needed tension placed on the layer or web during manufacturing of the first barrier layer and/or the absorbent article including the first barrier layer. If the layer or web cannot be adequately tensioned during manufacturing, this results in creasing of the layer and/or tearing of the layer. Additionally, the layer or web needs to endure multiple transformations during the formation of the absorbent articles, such as the addition of multiple layers. Converting speeds for manufacturing absorbent hygiene products exceeds 100 meters/min. By contrast, traditional paper converting speeds are much lower, such as about 70 meters/min. Typically, converting lines for absorbent articles run at least 1.5 times faster than traditional paper converting speeds. Thus, conventional Kraft papers and other insufficiently extensible materials are not sufficiently stretchable to survive the folding, transporting, and sealing operations used in the production of absorbent hygiene products without wrinkling, tearing, or web slack, which results in productivity losses, increased scrap, and generally low quality final products.

The first barrier layer 23 may have a basis weight of about 30 gsm to about 70 gsm, or from about 35 gsm to about 65 gsm, or from about 45 gsm to about 60 gsm, or about 48 gsm, according to the Basis Weight test method disclosed herein. In some embodiments, the first barrier layer 23 includes a paper. The paper having lower basis weights or basis weights less than 30 gsm, according to the Basis Weight test method disclosed herein, may not have sufficient strength to withstand the creping process and/or the forces exerted during normal wear of the absorbent article.

It is to be appreciated that the stretch direction of the barrier layer may be based on orientation of the layer with respect to the user. For example, the stretch direction of the first barrier layer may be substantially parallel to the longitudinal axis L of the absorbent article such that it provides stretch along the longitudinal axis during use, such as when the user sits or engages in activity the barrier layer may stretch accordingly and not break or bunch. It has also been found that other types of users may require stretch is a direction substantially perpendicular to the longitudinal axis L of the absorbent article. For example, it may be more consumer preferred to have the first barrier layer stretch in a direction substantially parallel to the transverse direction to aid in maintaining better conformity to the user's body during use, such as sitting, standing, and performing activities, and to prevent bunching and tearing of the layer.

The first barrier layer 23 should also be flexible to better conform to the body of the user. Absorbent articles that conform more closely and/or more easily to the body are perceived as being more comfortable by the user. To achieve this flexibility, the first barrier layer 23 may have a machine direction flexural modulus of less than about 900 N/mm² or less than about 700 N/mm² or less than about 500 N/mm² or less than about 250 N/mm² or less than about 200 N/mm², according to test method. Additionally, or alternatively, the first barrier layer 23 may have a cross direction flexural modulus of less than about 900 N/mm² or less than about 700 N/mm² or less than about 500 N/mm² or less than about 250 N/mm² or less than about 200 N/mm², according to test method. As discussed in greater detail below, it has been found that creped paper provides better flexibility than non-creped papers.

As previously discussed, users strongly prefer absorbent articles that are discrete. One of the challenges of using having a fluid barrier layer that is made from materials that do not contain synthetic polymers and films is noise. For example, paper is a relatively noisy material as compared to polymeric fibers and films, which are traditionally used in barrier layers. It has been found that having a first barrier layer that is void of synthetic fibers and films having an average loudness of less than about 60 dB or less than about 58 dB or less than about 55 dB or less than about 50 dB allows the product to be used and maintain discreteness due to a relatively lower level of noise. To achieve these lower noise levels for paper, for example, the paper may be creped, which allows for greater flexibility allowing for noise to be reduced, and/or coated. As described below, it has been found that creped and coated paper provides better sufficient noise reduction such that the level is within about 10 dB of a plastic film. It is to be appreciated that additional noise reduction may be possible by joining the first barrier layer to adjacent layers such at the core and/or the second barrier layer.

In some embodiments, the first barrier layer may include a regenerated cellulose film.

### Second Barrier Layer—Backsheet

The absorbent article 10 may include a second barrier layer 14. It is to be appreciated that the absorbent article 10 need not include a second barrier layer 14 where the first barrier layer is sufficient to contain the maximum amount of fluid designed for the particular absorbent article. However, the second barrier layer 14 may be used as an additional barrier to prevent leakage of fluid, and/or for structural purposes to retain the placement of the absorbent article and/or the layer(s), and/or to have a more aesthetically pleasing product by having an outer layer that is soft and flexible. The second barrier layer 14 may be positioned adjacent to the first barrier layer 23, such as illustrated in FIGS. 2A and 2B. The second barrier layer 14 may be joined to at least one of the topsheet layer 12, the absorbent core layer 15, and the first barrier layer 23. The second barrier layer 14 may be peripherally joined or partially or fully joined to some other portion of the absorbent article 10. The second barrier layer 14 may include a garment-facing surface and a wearer-facing surface. The garment-facing layer of the second barrier layer may be disposed on the user's garment and serve as the final barrier for fluid or an aesthetically pleasing layer that is externally facing.

The second barrier layer 14 may be substantially free of or void of synthetic materials, such as synthetic fibers and films, to achieve an absorbent article with a relatively reduced adverse environmental impact. The second barrier layer 14 may include, consist essentially of, or consist of cellulosic material. The cellulosic material may include, consist essentially of, or consist of fibers of pulp, cotton, flax, hemp, jute, kenaf, regenerated cellulose (such as rayon, which may be viscose or lyocell), or mixtures thereof. For example, the second barrier layer 14 may be a nonwoven material including cellulosic fibers. The second barrier layer 14 may have a basis weight of from about 30 gsm to about 60 gsm and a caliper of from about 0.25 to about 1.0 mm at 0.5 kPa. The second barrier layer 14 may have an acquisition time of greater than about 3 seconds, and a rewet of less than 1.5 g. according to standard test method EDANA NWSP 80.10 (09). The second barrier layer has a repellency of at least 20 seconds and 10% of Pore of less than about 175 micron or less than about 150 microns. However, it is to be appreciated that these values are based on the first barrier layer properties and may change in view of the first barrier layer properties, which will be discussed in greater detail with respect to the examples.

In some embodiments, the second barrier layer 14 may be the same as the first barrier layer 23. The second barrier layer 14 may have properties the same as or similar to those of the first barrier layer 23. Accordingly, in some embodiments, the absorbent article 10 may include a first barrier layer 23 including creped paper and a second barrier layer 23 including creped paper. In some embodiments, the first barrier layer 23 may include a nonwoven and the second barrier layer 12 may include a nonwoven.

The second barrier layer 14 may be different from the first barrier layer 23. For example, in instances where the first barrier layer 23 is sufficient to prevent the leakage of fluid for normal, intended levels for that absorbent article, the second barrier layer 14 need only provide minimal barrier protected or may not be used for its barrier properties but rather for structural purposes, such as keeping the first barrier layer in the proper position or enclosing the absorbent core layer, the fluid management layer, and the first barrier layer between the topsheet layer and second barrier layer. For example, the second barrier layer may include cellulosic fibers. These cellulosic fibers may be hydrophobic and/or hydrophilic. For example, the second barrier layer may include one or more stratum of hydrophobic rayon fibers. In some embodiments, the second barrier layer may include cotton. As previously discussed, the cotton fibers may be at least one or mechanically and chemically cleaned. Depending on the type and extent of the cleaning, the cotton fibers may be naturally hydrophobic. Overall, the second barrier layer may offer additional protection for the user when the layer is hydrophobic. The second barrier layer may be hydrophilic because the fibers that make up the layer are hydrophobic or a coating makes the layer hydrophobic, independent of whether the individual fibers are hydrophobic or hydrophilic. The coating may be a natural wax or a hydrophobic natural polymer such as lignin, casein, natural polyesters, or other natural hydrophobic materials.

### Layer Attachment

Two or more layers of the absorbent article 10 may be joined. Each of the layers may be joined to one or more layers. The layers may be joined by mechanical means, such as bonding, and/or chemical means, such as by an adhesive. For example, the layers may be joined by a uniform continuous layer of adhesive, a patterned layer of adhesive, or an array of separate lines, spirals, or spots of adhesive. The two or more layers may be joined using heat bonds, pressure bonds, ultrasonic bonds, dynamic mechanical bonds, or any other suitable attachment methods or combinations of these attachment methods as are known in the art. The adhesive may be at least one of hotmelt adhesives, pressure-sensitive adhesives, water-based adhesive, solvent-based adhesives, curable adhesives, and sealants.

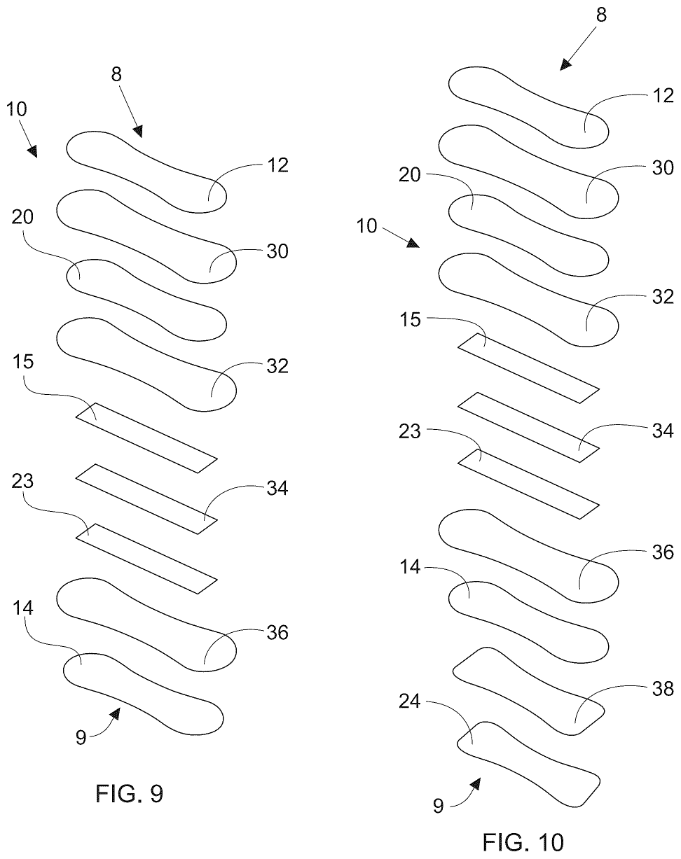

For example, as illustrated in FIG. 9, the one or more layers may each be going by an adhesive. The topsheet layer 12 may be joined by a first adhesive 30 to the fluid management layer 20; the fluid management layer 20 may be joined by a second adhesive 32 to the absorbent core layer 15; the absorbent core layer 15 may be joined by a third adhesive 34 to the first barrier layer 23; and the first barrier layer 23 may be joined by a fourth adhesive 36 to the second barrier layer 12, when present. As previously discussed, each of the first, second, third, and fourth adhesives may be the same or different in terms of composition of materials, application method, and application pattern.

In some embodiments, the absorbent article 10 may include a release cover 24, such as illustrated in FIG. 10. The release cover 24 may be joined to at least one of the first barrier layer 23 and the second barrier layer 12. The release cover 24 may serve to protect an attachment adhesive 38 until the user wants to expose the adhesive to attach the absorbent article 10 to an undergarment. The release cover 24 may include cellulosic material, such as paper. The release cover may be a silicone coated paper, a plastic film, or any other easily removable cover. The release cover may be in a single piece or in a multitude of pieces, e.g., to cover the individual adhesive areas. It also can perform other functions such as providing individualized packaging for the article or provide a disposal function. Any commercially available release cover or film may be used. Suitable examples include BL 30 MG-A SILOX EI/O, BL 30 MG-A SILOX 4 P/O available from Akrosil Corporation, and M&W films available from Gronau in Germany, under the code X-5432.

In some embodiments, the first, second, third, and fourth attachment adhesives may be void of synthetic polymers.

### Combination of Layers

The topsheet layer and at least one of the first barrier layer and the second barrier layer may be joined together to form an outer periphery of the disposable absorbent article. A periphery of the absorbent core and/or the fluid management layer may be disposed inboard of the outer periphery. For example, the absorbent core may have end edges extending generally parallel to a transverse axis and side edges extending generally parallel to a longitudinal axis. Each of the end edges and side edges may be disposed inboard of the outer periphery. Similarly, the fluid management layer may comprise end edges which extend generally parallel to the transverse axis and side edges which extend generally parallel to the longitudinal axis. The end edges and side edges may be disposed inboard of the outer periphery. Alternatively, the end edges may be coterminous with the outer periphery to the extent that the end edges intersect the outer periphery. In addition, or independently of the end edges of the fluid management layer, the side edges of the fluid management layer may be coterminous with the outer periphery of the absorbent article.

Additionally, the end edges and/or side edges of the absorbent core and/or fluid management layer may be curvilinear in nature. For example, the side edges of the absorbent core and/or the fluid management layer may curve inward from the ends toward the transverse axis. Such construction may help with conformity of the absorbent article. Similarly, the end edges in conjunction with or independently of the side edges of the absorbent core and/or fluid management layer may comprise a curvilinear path which is either generally concave or generally convex.

The layers may be joined by any suitable means, including for example adhesive bonding, mechanical bonding, ultrasonic bonding, and combinations thereof. Bonding may be continuous or discontinuous.

The inventors have surprisingly found the combination of layers of the present disclosure, results in a soft and dry feeling article that retains its shape despite compression and friction during wear while being void of synthetic fibers and films and having one or more layers that are substantially free of synthetic materials, which may include fibers, films, and adhesives. As mentioned above, the topsheet may comprise a plurality of macro deformations, which may be in the form of apertures and/or recessions, which may reduce contact area between the article and the wearer's skin and thereby reduce the likelihood of sticking, abrasion, or wet feel. Further, one or more features may provide porosity to the article, providing an airy and comfortable feeling to the wearer. In nonlimiting examples, the topsheet, fluid management layer and/or absorbent core may be void of synthetic fibers and film materials, as previously discussed.

As mentioned, absorbent articles of the present invention should be comfortable for the user to wear, which is particularly important where articles are liners which are worn for long periods of time and on a daily basis. Indeed, many individuals wear a liner for 8 hours or more. As such, liner wearers desire an article that will not stick to the skin, deform and remains dry feeling to the wearer. Moreover, wearers having a high body mass index (i.e., a BMI of at least 30) desire products that can maintain their shape and withstand high compression, regardless of the duration of wear. Articles of the present invention may address these needs.

An article of the present disclosure provides sufficient integrity to prevent bunching, i.e., rolling or folding in the x-y direction due to compression of the legs during wear while maintaining sufficient flexibility to allow the product to better conform to the wearer's body and to be more comfortable during wear. The absorbent article has one or more layers that allow the liner to maintain flexibility despite being made from cellulosic material that is generally stiffer than traditional synthetic materials used in traditional absorbent articles. Flexibility is achieved by the methods used to produce the absorbent article. The following examples demonstrate those cellulosic materials that achieve the desired flexibility so that the absorbent article is comfortable for the wearer and also sufficiently handled the fluid to prevent leaks and maintain a dry feeling.

To maintain a dry-feeling for the wearer, the absorbent article may exhibit an acquisition time of less than about 5 seconds, less than about 3 seconds, or less than about 2.5 seconds, or less than about 1.5 seconds or from about 1 second to about 2.5 seconds, or from about 1.5 seconds to about 2.2 seconds when measured in accordance with the Liquid Acquisition Time test method described herein.

Additionally, or alternatively, the article may exhibit Standard Rewet of about 0.2 g or less, or about 0.15 g or less, or about 0.1 g or less, or undetectable, according to the Standard Rewet test method.

Disposable absorbent articles according to the present invention were constructed and tested. Additionally, comparative example disposable absorbent articles were purchased and tested. For each of the Inventive Examples 1-3, the liner was constructed of a topsheet layer having a generally hour-glass shape or peanut-like shape, a fluid management layer having a generally hour-glass shape or peanut-like shape, an absorbent core layer having a rectangular shape, a first barrier layer having a generally rectangular shape, and a second barrier layer having an hour-glass or peanut-like shape, such as illustrated in FIG. 2A. The fluid management layer was larger than each of the absorbent core layer and the first barrier layer. The fluid management layer was smaller than the topsheet layer. Each of the layers were attached to the adjacent layer by adhesive. The topsheet layer and the second barrier layer were joined about the perimeter of the liner. The additional details of the layers included in each of Inventive Examples 1-3 are described as follows.

Comparative Example 1 includes synthetic fibers and films and is not substantially free of synthetic materials. Comparative Example 1 includes a topsheet including 100% organic cotton and having a basis weight of 35 gsm, which is available as CO35W from Daiwabo Holdings Company, Ltd.; a fluid management layer including a multibonded airlaid layer (MBAL) having a basis weight of 60 gsm and comprising pulp fibers and bicomponent fibers, which is available from Glatfelter Corporation; an absorbent core layer including a multibonded airlaid layer (MBAL) comprising pulp fiber, bicomponent fibers, and absorbent gelling materials and having a basis weight of 160 gsm, which is available from Glatfelter Corporation; a first barrier layer including a non-breathable, polyethylene film having a basis weight of 19 gsm, which is available from Exten; and a second barrier layer including a spunbond polypropylene film having a basis weight of 18 gsm, which is available from PFNonwovens Holding s.r.o. Comparative Example 1 is a marketed product and is available in Europe under the name ALWAYS® Daily Liners, size normal, with 100% pure cotton top layer.

Comparative Example 2 includes synthetic fibers and films and is not substantially free of synthetic materials. More specially, the barrier layer is made from synthetic material. Comparative Example 2 includes a topsheet including a 100% organic cotton layer and an absorbent core layer including pulp fibers. Comparative Example 2 includes a single barrier layer including a spunbond polypropylene layer. Comparative Example 2 does not include a fluid management layer. The liner is available in Germany under the name COSMEA® Bio-Slipeinlagen Normal and is manufactured by W. Pelz GmbH & Co.

Comparative Example 3 is void of synthetic fibers and films. Comparative Example 3 includes a topsheet having a basis weight of 35 gsm and being a spunlace nonwoven having 100% organic cotton fibers and having apertures therethrough. The cotton fibers are hydrophilic. Such a topshect layer is available as CO35W from Daiwabo Holdings Company, Ltd. The fluid management layer has a basis weight of 55 gsm and is a conventionally made spunlace layer having 70% 1.7 dTex lyocell fibers and 30% mechanically cleaned cotton, by weight. The fluid management layer has a moment of inertia of 923 μm⁴ and is coated with a hydrophilic coating. The absorbent core layer is a hydrogen bonded airlaid layer (HBAL) including pulp fibers and having a basis weight of 170 gsm. Such an absorbent core layer is available as T-169-S-TCF from McAirlaid's Inc. The first barrier layer is a creped paper including a wax coating and having a basis weight of 48 gsm, available from Pelta Medical Papers. The second barrier layer is a spunlace nonwoven layer comprising mechanically cleaned, hydrophobic cotton fibers and having a basis weight of 40 gsm.

Inventive Example 1 is void of synthetic fibers and films. Inventive Example 1 includes a topsheet having a basis weight of 35 gsm and being a spunlace nonwoven having 100% organic cotton fibers and having apertures therethrough. The cotton fibers are hydrophilic. Such a topsheet layer is available as CO35W from Daiwabo Holdings Company, Ltd. The fluid management layer has a basis weight of 60 gsm and is a hydrogen bonded airlaid layer (HBAL) having pulp fibers. Such a fluid management layer is available as T-060-S-TCF from McAirlaid's Inc. The absorbent core layer is a 170 gsm hydrogen bonded airlaid layer (HBAL) including pulp fibers. Such an absorbent core layer is available as T-169-S-TCF from McAirlaid's Inc. The first barrier layer is a creped paper including a wax coating and having a basis weight of 48 gsm. The second barrier layer is a spunlace nonwoven layer comprising mechanically cleaned, hydrophobic cotton fibers and having a basis weight of 40 gsm.

Inventive Example 2 is void of synthetic fibers and films. Inventive Example 2 includes a topsheet having a basis weight of 35 gsm and being a spunlace nonwoven having 100% organic cotton fibers and having apertures therethrough. The cotton fibers are hydrophilic. Such a topsheet layer is available as CO35W from Daiwabo Holdings Company, Ltd. The fluid management layer has a basis weight of 55 gsm and is a spunlace layer having 25% 3.3 dTex trilobal viscose fibers and 75% 6.4 dTex round viscose fibers, by weight, and has a caliper of about 0.74 mm. The fluid management layer has a moment of inertia of 13200 μm⁴ and is coated with a hydrophilic coating. The absorbent core layer is a 170 gsm airlaid absorbent core including pulp fibers. Such an absorbent core layer is available as T-169-S-TCF from McAirlaid's Inc. The first barrier layer is a creped paper including a wax coating and having a basis weight of 48 gsm. The second barrier layer is a spunlace nonwoven layer comprising mechanically cleaned, hydrophobic cotton fibers and having a basis weight of 40 gsm.

Inventive Example 3 is void of synthetic fibers and films. Inventive Example 3 includes a topsheet having a basis weight of 35 gsm and being a spunlace nonwoven having 4.2 dTex hydrophobic Olea fibers, available from Kelheim Fibres GmbH, and having apertures therethrough. The fluid management layer has a basis weight of 55 gsm and is a spunlace layer having 40% 3.3 dTex trilobal viscose fibers and 60% 4.2 dTex Olea fibers, by weight, available from Kelheim Fibres GmbH, and has a caliper of about 0.87 mm. The fluid management layer has a moment of inertia of 7800 μm⁴ and is coated with a hydrophilic coating. The absorbent core layer is an airlaid absorbent core including pulp fibers and having a basis weight of 170 gsm. Such an absorbent core layer is available as T-169-S-TCF from McAirlaid's Inc. The first barrier layer is a creped paper including a wax coating and having a basis weight of 48 gsm. The second barrier layer is a spunlace nonwoven layer comprising mechanically cleaned, hydrophobic cotton fibers and having a basis weight of 40 gsm.

For each of Inventive Examples 2 and 3, the fluid management layer was produced using a spunlace process as described in US Pat. Publication Nos. 2020/0315873 and 2020/0315874. Using this process results in a fluid management layer that has increased caliper due to the reduced stretching and compression of the layer while being manufactured Inventive Examples 2 and 3 have a caliper of from about 0.67 mm to about 0.96 mm, as determined by the Caliper test method disclosed herein. Inventive Examples 2 and 3 have a caliper factor of from about 0.12 to about 0.16. In comparison, conventionally manufactured spunlace materials have a caliper factor of less than about 0.09. The caliper factor is the caliper per 10 gsm of basis weight of the sample. The caliper factor is calculated as caliper/(basis weight/10), where the caliper is determined by the Caliper method disclosed herein and the basis weight is calculated by the Basis Weight test method disclosed herein.

As previously discussed, the liner should be such that it is relatively thin and comfortable to wear and keeps the user feeling relatively dry. It is to be appreciated that the absorbent core may be used to overcompensate for the failure of the other layers to acquire and hold fluid. However, a bulky and overdesigned absorbent core layer will not be comfortable for the user to wear for extended periods of time and unnecessary increases cost of the product. The examples demonstrate a liner having a caliper of less than about 3.0 mm or less than about 2.5 mm or about 2.0 mm and achieves the desired acquisition time and capacity while being void of synthetic fibers and films and/or has one or more layers that are substantially free of synthetic materials, such as fibers, films, and adhesives.

Comparative Example 1 is included to capture a product that is not void of synthetic fibers and film and has the traditional fluid handling capabilities of such a product. Comparative example 1 is included as a baseline for fluid handling performance for a product including synthetic materials. Comparative Example 2 is a product that is currently available in the market that includes certain layers that are void of synthetic fibers and films but still includes a synthetic backsheet or second barrier. By contrast, Comparative Example 3 is void of synthetic fibers and films but includes a fluid management layer that lacks the required structure to achieve the moment of inertia as specific herein.

As demonstrated in Table 2 below, Inventive Example 1 is an absorbent article that is void of synthetic fibers and films. Inventive Example 1 has an acquisition time of less than about 0.5 seconds or about 0.4 seconds according to the Liquid Acquisition Time test method disclosed herein. Although this is longer than Comparative Example 1, which is not void of synthetic fibers and films, this acquisition time is sufficient for the article to adequately handle fluid and maintain a relatively dry feeling. Comparative Example 2 includes a topsheet and fluid management layer that are void of synthetic fibers and films but includes a synthetic barrier layer. As demonstrated in Table 2, Comparative Example 2 has a significantly higher acquisition time and thus does not handle fluid as well as Inventive Example 1. Comparative Example 2 has increased likelihood that it will leak because fluid is not absorbed quickly as compared to Inventive Example 1. Additionally, Comparative Example 2 has a higher rewet value, which was determined by the Standard Rewet method disclosed herein, than Inventive Example 1. Further, although the absorbent capacity, determined by the Dunk Capacity test method herein, of Comparative Example 2 is greater than Inventive Example 1, Comparative Example 2 will likely feel less dry due to the higher rewet value and the longer acquisition time. Inventive Example 1 is also thinner or has a caliper that is less than the caliper of Comparative Example 1 and thus, should be more comfortable to wear due to the lower caliper. In summary, Inventive Example 1 perform almost as well as Comparative Example 1 and better than Comparative Example 2. It has been found that for an article being void of synthetic fibers and films and/or substantially free of synthetic materials (such as fibers and films) having a fluid management layer including airlaid pulp fibers that are hydrogen bonded and a topsheet layer including cotton exhibits the required fluid handling properties when the acquisition time is less than about 1 second according to the Liquid Acquisition Time test method disclosed herein and a rewet of less than about 0.20 g.

TABLE 2

| | | | | Finished |
|---|---|---|---|---|
| | Liquid | | | Product, |
| | Acquisition | | Absorbent | Liner |
| | Time | Rewet | Capacity | Caliper |
| Example | (seconds) | (g) | (g) | (mm) |
| Comparative Example 1 | 0.25 | 0.008 | 13.2 | 2.3 |
| Comparative Example 2 | 2.11 | 0.224 | 18.0 | 2.7 |
| Inventive Example 1 | 0.42 | 0.105 | 10.4 | 2.0 |

As previously discussed, the wearer prefers an absorbent article that is relatively thinner and drier feeling. To obtain a liner that satisfies these criteria, the topsheet and fluid management layer may be optimized while also being void of synthetic fibers and films. Table 3 includes the data for various examples having the same first barrier layer, the same second barrier layer, and the same absorbent core layer. However, each of the examples including in Table 3 have various topsheet layers and fluid management layers. Each of the examples listed in Table 3 (Inventive Examples 2 and 3 and Comparative Example 3) have an absorbent capacity of about 10 g or less than about 11 g, according to the Dunk Capacity test method disclosed herein, and a finished product caliper of less than about 2.2 mm, according to the Caliper test method disclosed herein. Further, each of the examples listed in Table 3 includes a fluid management layer including rayon, such as Olea fibers and viscose fibers. A fluid management layer including rayon fibers improves the fluid handling properties of the absorbent article by having generally lower acquisition time and rewet, as compared to a fluid management layer including airlaid pulp fibers. Further, as demonstrated by the data included in Table 3, by increasing the moment of inertia of the fluid management layer, the acquisition time can be improved, as evidenced by Comparative Example 3 and Inventive Example 2. By having an absorbent article with a topsheet layer that is apertured and including hydrophobic rayon fibers and a fluid management layer including rayon fibers and having a moment of inertia greater than 3,975 μm⁴, the rewet may be further improved and the acquisition time maintained. It is to be appreciated that although rewet correlates to the feeling of dryness, the type of fluid discharges that a wearer experiences might vary, and sometimes a relatively large gush may occur, so the product should be able to handle this relatively large gush by absorbing it relatively quickly to prevent liquid from rolling over the product surface and leaking, which is especially important during the wearer's movement. Inventive example 2 with higher MOI has demonstrated faster acquisition, which prevents against such leaks, in comparison to the comparative example with lower a MOI that has relatively slower acquisition (even though the feeling of dryness over a longer period of time might be the same for both examples). Also, for Inventive 3 that use of the hydrophobic topsheet additionally improves dryness experience, while still maintaining satisfactory acquisition time. It has been found that liners having a fluid management layer that exhibits a moment of inertia of great than about 2,000 μm⁴ and an acquisition time of less than about 0.4 seconds and a rewet of less than about 0.1 g, has fluid management that allows the liner to be comfortable and dry feeling.

TABLE 3

| | Topsheet | Moment of | First | |
|---|---|---|---|---|
| | Layer & Fluid | Inertia | Liquid | |
| | Management | (μm⁴) - Fluid | Acquisition | |
| | Layer Summary | Management | Time | Rewet |
| Example | Description | Layer | (seconds) | (g) |
| Comparative | Topsheet = | 923 | 0.43 | 0.054 |
| Example 3 | Hydrophilic | | | |
| | Cotton fibers | | | |
| | Fluid Management | | | |
| | Layer = Rayon | | | |
| | (lyocell) fibers & | | | |
| | Cotton fibers | | | |
| Inventive | Topsheet = | 13200 | 0.13 | 0.055 |
| Example 2 | Hydrophilic | | | |
| | Cotton Fluid | | | |
| | Management | | | |
| | Layer = Rayon | | | |
| | (viscose) fibers | | | |
| Inventive | Topsheet = Olea | 7800 | 0.28 | 0.003 |
| Example 3 | fibers* | | | |
| | Fluid Management | | | |
| | Layer = Olea | | | |
| | fibers* and trilobal | | | |
| | viscose fibers | | | |

*Olea fibers are a type of Rayon fiber that are made hydrophobic and are available from Kelheim Fibres GmbH

As previously discussed, the fluid that may not remain trapped by the topsheet layer, the fluid management layer, and/or the absorbent core layer needs to be prevented from causing leaks onto the undergarments of the wearer. At least one of the first barrier layer and the second barrier layer are used to prevent fluid from leaking onto the wearer's undergarments. Generally, the greater the amount of fluid that is released from the absorbent core layer through, for example, compression of the core under pressure, the more likely that fluid will leak from the absorbent core layer and need to be handled by one or more barrier layers. To demonstrate the necessary parameters of the first barrier layer and/or the second barrier layer, disposable absorbent articles according to the present disclosure were constructed and tested. Generally, it has been found that by controlling the pore size and repellency of the barrier layer, which may be the first barrier layer and/or the second barrier layer, the entry points for fluid to penetrate into the layer and the forces that deter fluid from advancing through the layer may be optimized, which deters fluid from advancing through the barrier layer(s). Further, to evaluate these parameters, the Caliper Flow Porometry test method disclosed herein was used to determine the largest pore diameter, smallest pore diameter and mean pore diameter, and the Water Repellency test method disclosed herein was used to determine the repellency time for the barrier layer(s) as a measure of the layer's hydrophobicity. The liner was also evaluated for wet through or, stated another way, for the ability of fluid that is released from the absorbent core layer to leak from the absorbent article. The wet through was determined by the Wet Through test method disclosed herein.

It has been found that at least one of the first barrier layer and the second barrier layer should have a 10% of Pore value, as identified by the Caliper Flow Porometry test method, that is less than about 20 microns or, more preferably, that is less than about 15 microns, and a repellency time, as determined by the Water Repellency test method, of greater than about 5 seconds or, more preferably, greater than about 10 seconds. The barrier layer has a plurality of pores having a distribution of pore diameter. The distribution of pore diameter may be 10% of pores are those pores that are larger than 90% of the distribution of pore diameters that are smaller. For example, the first barrier layer has a repellency of greater than 5 seconds and a 10% of Pore value of less than 20 microns or less than 15 microns, and the second barrier layer has a repellency of greater than 20 seconds and a 10% of Pore value of less than 175 microns or less than 150 microns, as determined by the Caliper Flow Porometry and Water Repellency test methods. It is to be appreciated that these two barrier layers work together and may be selected in view of one another, as demonstrated in the examples.

As discussed herein, the liner may include a first barrier layer and a second barrier layer. It has been found that to deter leakage of fluid, the first barrier layer should include a large pore diameter that is less than about 20 microns or, more preferably, less than about 15 microns and a repellency time of greater than about 5 second or, more preferably, greater than about 10 seconds. This first barrier layer may be disposed on a second barrier layer. The second barrier layer should include a large pore diameter of less than about 175 microns or, more preferably, less than about 150 microns and have a repellency time of less than about 20 seconds. The combination of the first barrier layer and the second barrier layer as specified has been found to adequately handle fluid that may be released from the absorbent core layer.

The following examples as outlined in Tables 4 and 5 demonstrate various embodiments of a liner and the ability to handle fluid based on wet through and the properties of the barrier layer(s). Table 3 includes the details of each of the topsheet layer, fluid management layer, absorbent core layer, first barrier layer, and second barrier layer. For each of the examples 1-B through 10-B, the liner was constructed of a topsheet layer having a generally hour-glass shape or peanut-like shape, a fluid management layer having a generally hour-glass shape or peanut-like shape, an absorbent core layer having a rectangular shape, a first barrier layer having a generally rectangular shape, and a second barrier layer having an hour-glass or peanut-like shape, such as illustrated in FIG. 2A. The fluid management layer was larger than each of the absorbent core layer and the first barrier layer. The fluid management layer was smaller than the topsheet layer. Each of the layers were attached to the adjacent layer by adhesive. The topsheet layer and the second barrier layer were joined about the perimeter of the liner.

TABLE 4

| | Details of Topsheet Layer, | |
|---|---|---|
| | Fluid Management Layer & | Details of First Barrier Layer |
| Example | Absorbent Core Layer | and Second Barrier Layer |
| 1-B | Topsheet layer: hydrophilic 100% | First Barrier layer: PeltaMed |
| | organic cotton layer with a basis | Lightweight Creped Waxed Paper |
| | weight of 35 gsm, available as | having a basis weight of 48 gsm, |
| | CO35W from Daiwabo Holdings | available from Pelta Medical Papers |
| | Company, Ltd. | Second Barrier layer: mechanically |
| | Fluid Management Layer: hydrogen | cleaned, hydrophobic cotton spunlace |
| | bonded airlaid (HBAL) layer with | nonwoven layer with a basis weight |
| | a basis weight of 60 gsm, available | of 40 gsm |
| | as T-060-S-TCF from McAirlaid's Inc. | |
| | Core layer: hydrogen bonded airlaid | |
| | (HBAL) core layer with a basis | |
| | weight of 170 gsm, available as T- | |
| | 169-S-TCF from McAirlaid's Inc | |
| 2-B | Topsheet layer: hydrophilic 100% | First Barrier layer: Natureflex 28 |
| | organic cotton layer with a basis | NP regenerated cellulose film, |
| | weight of 35 gsm, available as | available from Futamura Chemical |
| | CO35W from Daiwabo Holdings | UK Ltd. |
| | Company, Ltd. | Second Barrier layer: mechanically |
| | Fluid Management Layer: HBAL | cleaned, hydrophobic cotton spunlace |
| | layer with a basis weight of 60 | nonwoven layer with a basis weight |
| | gsm, available as T-060-S-TCF | of 40 gsm |
| | from McAirlaid's Inc. | |
| | Core layer: HBAL layer with a | |
| | basis weight of 170 gsm, | |
| | available as T-169-S-TCF from | |
| | McAirlaid's Inc | |
| 3-B | Topsheet layer: hydrophilic 100% | First Barrier layer: PeltaMed 455 |
| | organic cotton spunlace, available | Bleached Creped Waxed paper having |
| | as F091A0350 from Fibertex | a basis weight of 50 gsm, available |
| | Nonwovens | from Pelta Medical Papers |
| | Fluid Management Layer: spunlace | Second Barrier layer: mechanically |
| | nonwoven made from a blend of | cleaned, hydrophobic cotton spunlace |
| | GALAXY ® viscose fibers and | nonwoven layer with a basis weight |
| | OLEA ® viscose fibers from | of 40 gsm |
| | Keilheim Fibres, coated with a | |
| | hydrophobic coating and having | |
| | a basis weight of 55 gsm | |
| | Core Layer: HBAL layer having | |
| | a basis weight of 170 gsm, | |
| | available as T-169-S-TCF from | |
| | McAirlaid's Inc | |
| 4-B | Topsheet Layer: hydrophilic 100% | First Barrier layer: PeltaMed 455 |
| | organic cotton spunlace layer, | Bleached Creped Waxed paper having |
| | available as F091A0350 from | a basis weight of 50 gsm, available |
| | Fibertex Nonwovens | from Pelta Medical Papers |
| | Fluid Management Layer: spunlace | Second Barrier layer: JTEX 2003 HO |
| | nonwoven made from a blend of | hydrophobic viscose layer having |
| | GALAXY ® viscose fibers and | a basis weight of 30 gsm, available |
| | OLEA ® viscose fibers from | from Texol srl |
| | Keilheim Fibres, coated with a | |
| | hydrophobic coating and having | |
| | a basis weight of 55 gsm | |
| | Core Layer: HBAL layer having | |
| | a basis weight of 170 gsm, | |
| | available as T-169-S-TCF from | |
| | McAirlaid's Inc | |
| 5-B | Topsheet layer: hydrophilic cotton | First Barrier layer: PeltaMed |
| | layer with a basis weight of 35 | Lightweight Creped Waxed Paper |
| | gsm, available as CO35W from | having a basis weight of 48 gsm, |
| | Daiwabo Holdings Company, Ltd. | available from Pelta Medical Papers |
| | Fluid Management Layer: HBAL | Second Barrier layer: hydrophilic |
| | layer with a basis weight of 60 | 100% organic cotton layer having a |
| | gsm, available as T-060-S-TCF | basis weight of 35 gsm, available |
| | from McAirlaid's Inc. | as F091A0350 from Fibertex |
| | Core layer: HBAL layer with a | Nonwovens |
| | basis weight of 170 gsm, | |
| | available as T-169-S-TCF from | |
| | McAirlaid's Inc | |
| 6-B | Topsheet layer: hydrophilic 100% | First Barrier layer: PeltaMed |
| | organic cotton layer with a basis | Lightweight Creped Waxed Paper |
| | weight of 35 gsm, available as | having a basis weight of 48 gsm, |
| | CO35W from Daiwabo Holdings | available from Pelta Medical Papers |
| | Company, Ltd. | Second Barrier layer: PeltaMed |
| | Fluid Management Layer: HBAL | Lightweight Creped Waxed Paper |
| | layer with a basis weight of 60 | having a basis weight of 48 gsm, |
| | gsm, available as T-060-S-TCF | available from Pelta Medical Papers |
| | from McAirlaid's Inc. | |
| | Core layer: HBAL layer with a | |
| | basis weight of 170 gsm, | |
| | available as T-169-S-TCF | |
| | from McAirlaid's Inc | |
| 7-B | Topsheet layer: Apertured | First Barrier layer: Sulflex white |
| | spunlace nonwoven made from | vegetable parchment paper having |
| | OLEA Viscose fibers from Keilheim | a basis weight of 50 gsm, available |
| | Fibres and having a basis weight | from Ahlstrom |
| | of 35 gsm | Second Barrier layer: mechanically |
| | Fluid Management layer: spunlace | cleaned, hydrophobic cotton |
| | nonwoven made from a blend of | spunlace nonwoven layer with a |
| | GALAXY ® Viscose fibers and | basis weight of 40 gsm |
| | OLEA ® Viscose fibers from | |
| | Keilheim Fibres, coated with a | |
| | hydrophilic coating and having | |
| | a basis weight of 55 gsm | |
| | Core layer: HBAL layer with a | |
| | basis weight of 170 gsm, | |
| | available as T-169-S-TCF from | |
| | McAirlaid's Inc | |
| 8-B | Topsheet layer: hydrophilic | First Barrier layer: PeltaMed |
| | cotton layer with a basis | Lightweight Creped Waxed Paper |
| | weight of 35 gsm, available as | having a basis weight of 48 gsm, |
| | CO35W from Daiwabo Holdings | available from Pelta Medical Papers |
| | Company, Ltd. | Second Barrier layer: mechanically |
| | Fluid Management layer: Spunlace | cleaned, hydrophobic cotton |
| | nonwoven made from a blend of | spunlace nonwoven layer with a |
| | conventional viscose fibers and | basis weight of 40 gsm |
| | GALAXY ® Viscose fibers from | |
| | Keilheim Fibres coated with a | |
| | hydrophilic coating having a | |
| | basis weight of 55 gsm | |
| | Core layer: Two layers of | |
| | spunlace nonwovens including | |
| | Bramante viscose hollow fibers | |
| | from Kelheim Fibres GmbH and | |
| | each layer having a basis | |
| | weight of 85 gsm | |
| 9-B | Topsheet layer: hydrophilic | First Barrier layer: Natureflex |
| | 100% organic cotton layer with | 28 NP regenerated cellulose film, |
| | a basis weight of 35 gsm, | available from Futamura Chemical |
| | available as CO35W from Daiwabo | UK Ltd. |
| | Holdings Company, Ltd. | Second Barrier layer: mechanically |
| | Fluid Management layer: Spunlace | cleaned, hydrophobic cotton |
| | nonwoven made from a blend of | spunlace nonwoven layer with a |
| | conventional viscose fibers and | basis weight of 40 gsm |
| | GALAXY ® Viscose fibers from | |
| | Keilheim Fibres GmbH coated with | |
| | a hydrophilic coating having a | |
| | basis weight of 55 gsm | |
| | Core layer: Two layers of | |
| | spunlace nonwovens including | |
| | Bramante viscose hollow fibers | |
| | from Kelheim Fibres GmbH and | |
| | each layer having a basis | |
| | weight of 85 gsm | |
| 10-B | Topsheet layer: hydrophilic 100% | First Barrier layer: PeltaMed |
| | organic cotton layer with a basis | Lightweight Creped Waxed Paper |
| | weight of 35 gsm, available as | having a basis weight of 48 gsm, |
| | CO35W from Daiwabo Holdings | available from Pelta Medical Papers |
| | Company, Ltd. | Second Barrier layer: hydrophilic |
| | Fluid Management layer: Spunlace | 100% organic cotton layer having |
| | nonwoven made from a blend of | a basis weight of 35 gsm, |
| | conventional viscose fibers and | available as F091A0350 from |
| | GALAXY ® Viscose fibers from | Fibertex Nonwovens |
| | Keilheim Fibres, coated with a | |
| | hydrophilic coating and having | |
| | a basis weight of 55 gsm | |
| | Core layer: Two layers of spunlace | |
| | nonwovens including Bramante | |
| | viscose hollow fibers from Kelheim | |
| | Fibres GmbH and each layer having | |
| | a basis weight of 85 gsm | |

The likelihood of a product leaking, which is quantified by Wet Through for the Examples discussed herein, is based on the porosity of the layer(s) and the repellency. A layer having a distribution of relatively small pore sizes and a relatively high repellency, would lead to a product that is less likely to leak. The pore size for the layer should be small to prevent fluid from leaking through, but this should be balanced with breathability, which makes the product more comfortable to wear for long periods of time. With reference to Table 5 below, each of Examples 1-B through 3-B, 6-B and 7-B exhibit a Wet Through, as determined by the Wet Through test method disclosed herein, that would effectively prevent fluid from leaking onto the undergarment of a wearer. More specifically, each of Examples 1-B through 3-B, 6-B and 7-B have a Wet Through of less than 0.20 g. Each of Examples 1-B through 3-B, 6-B and 7-B have the required pore structure to prevent fluid from passing through the second barrier layer. In comparison, Examples 4-B and 5-B each have a relatively larger pore structure that would allow fluid to pass through the barrier layers. For instance, the pore structure should be small enough to prevent fluid from passing through one or more of the barrier layers to prevent leaks. As demonstrated by Examples 3-B and 4-B, the Wet Through increased by about 65% for Example 4-B as compared to Example 3-B and the largest pore increased in size by about 17.5% for Example 4-B as compared to Example 3-B. The increase in the pore structure resulted in significantly higher wet through. Additionally, as demonstrated by Examples 1-B and 5-B, Example 1-B included a hydrophobic second barrier layer and Example 5-B included a hydrophilic second barrier layer. Although each of Example 1-B and 5-B had the same first barrier layer, the difference in hydrophobicity, which is captured as repellency, caused the fluid for the hydrophilic second barrier layer to want to pull the fluid through the layer resulting in relatively greater wet through. By contrast, the hydrophobic second barrier layer worked to repel the fluid and prevent wet through as exhibited in Example 1-B. Further still, as demonstrated by Examples 1-B and 6-B, the first barrier layer and the second barrier layer may be the same, as in Example 6-B, or different, as in Example 1-B. Stated another way, the first barrier layer and the second barrier layer may have the same or different properties. Example 6-B has improved wet through as compared to Example 1-B because each of the first barrier layer and the second barrier layer have a relatively smaller pore structure.

Examples 8-B through 10-B demonstrate examples having a different absorbent core layer as compared to the other Examples. The absorbent core layer of Examples 8-B through 10-B was found to more easily release fluid upon being compressed by external forces, similar to those that occur when a wearer sits down or does relatively higher impact physical activity. The failure of the absorbent core layer to adequately hold the fluid then relied more heavily on the repellency and pore structure of each of the first barrier layer and the second barrier layer. As exhibited by Example 10-B, when the second barrier layer had a pore structure that was relatively large, the second barrier layer was unable to prevent fluid for passing through the layer and therefore resulted in higher wet through.

TABLE 5

| | First Barrier | | Second Barrier | | |
|---|---|---|---|---|---|
| | Layer | | Layer | | |
| | Porosity - | | Porosity - | | |
| | Largest Pore | | Largest Pore | | |
| | Diameter | | Diameter | | Wet Through, |
| | (microns) & | First Barrier | (microns) & | Second Barrier | Average & |
| | 10% of | Layer | 10% of | Layer | Standard |
| | Pore value | Repellency | Pore value | Repellency | Deviation |
| Example | (microns) | (seconds) | (microns) | (seconds) | (g) |
| 1-B | Largest pore = | 19.6 | Largest pore = | 25.2 | Avg: 0.139 g |
| | 8.63 μm | | 148.58 μm | | SD: 0.022 g |
| | 10% of Pore ≥ | | 10% of Pores ≥ | | |
| | 8.01 μm | | 144.72 μm | | |
| 2-B | Largest pore < | 10.4 | Largest pore = | 25.2 | Avg: 0.161 g |
| | 0.424 μm | | 148.58 μm | | SD: 0.005 g |
| | 10% of Pores < | | 10% of Pores ≥ | | |
| | 0.424 μm | | 144.72 μm | | |
| 3-B | Largest pore = | 23.6 | Largest Pore = | 27.1 | Avg: 0.180 g |
| | 15.87 μm | | 200.54 μm | | SD: 0.021 g |
| | 10% of Pores ≥ | | 10% of Pores ≥ | | |
| | 13.64 μm | | 140.08 μm | | |
| 4-B | Largest pore = | 23.6 | Largest pore = | 21.5 | Avg: 0.297 g |
| | 15.87 μm | | 171.72 μm | | SD: 0.087 g |
| | 10% of Pores ≥ | | 10% of Pores ≥ | | |
| | 13.64 μm | | 164.68 μm | | |
| 5-B | Largest pore = | 19.6 | Largest pore = | <1 | Avg: 0.267 g |
| | 8.63 μm | | 216.2 μm | | SD: 0.056 g |
| | 10% of Pores ≥ | | 10% of Pores ≥ | | |
| | 8.01 μm | | 189.92 μm | | |
| 6-B | Largest pore = | 19.6 | Largest pore = | 19.6 | Avg: 0.120 g |
| | 8.63 μm | | 8.63 μm | | SD: 0.011 g |
| | 10% of Pores ≥ | | 10% of Pores ≥ | | |
| | 8.01 μm | | 8.01 μm | | |
| 7-B | Largest pore < | 20.6 | Largest Pore = | 27.1 | Avg: 0.120 g |
| | 0.424 μm | | 200.54 μm | | SD: 0.002 g |
| | 10% of Pores < | | 10% of Pores ≥ | | |
| | 0.424 μm | | 140.08 μm | | |
| 8-B | Largest pore = | 19.6 | Largest pore = | 25.2 | Avg: 0.107 g |
| | 8.63 μm | | 148.58 μm | | SD: 0.014 g |
| | 10% of Pores > | | 10% of Pores > | | |
| | 8.01 μm | | 144.72 μm | | |
| 9-B | Largest pore < | 10.4 | Largest pore = | 25.2 | Avg: 0.153 g |
| | 0.424 μm | | 148.58 μm | | SD: 0.109 g |
| | 10% of Pores < | | 10% of Pores > | | |
| | 0.424 μm | | 144.72 μm | | |
| 10-B | Largest pore < | 10.4 | Largest pore = | <1 | Avg: 0.474 g |
| | 0.424 μm | | 216.2 mic | | SD: 0.098 g |
| | 10% of Pores < | | 10% of Pores > | | |
| | 0.424 μm | | 189.92 mic | | |

In addition to adequately handling fluid and preventing leakage of fluid, user or wearer also require the liner to be comfortable to wear for short and long periods of time. The traditional synthetic fibers and films used to make the various layers of the absorbent article were typically flexible and could easily conform to the wearer for a good fit. By contrast, paper is typically a stiffer product and does not conform as easily to wearer for a good fit. Thus, the barrier layer should be void of synthetic fiber and films or substantially free of synthetic materials, have good barrier properties, and be flexible to provide a comfortable fit. It has been found that certain papers have a lower flexural modulus, which translates generally into more flexibility. For example, Parchment paper and Kraft paper may not provide the needed flexibility to be comfortable during use. However, creped paper, such as that used in Examples 1-B and 3-B, can have the desired barrier properties or fluid handling properties and the needed flexibility. It has been found that a barrier layer having a Flexural Modulus of less than about 1000 N/mm² or less than bout 750 N/mm² or less than about 500 N/mm² or less than about 200 N/mm² in each of the cross direction and machine direction, or stated another way, the longitudinal direction and the transverse direction, has the needed flexibility. Table 6 includes several examples of various paper materials in comparison to the traditional plastic film. As demonstrated in Table 6, the crepe paper has a cross direction (CD) flexural modulus and a machine direction (MD) flexural modulus of less than 200 N/mm² and less than that of the plastic film. The cross direction (CD) flexural modulus and a machine direction (MD) flexural modulus are calculated using the Three Point Bend test method disclosed herein. As exhibited, creping the paper results in increased flexural modulus in each of the cross direction and machine direction.

TABLE 6

| | | Stiffness | Flexural | Stiffness | Flexural |
|---|---|---|---|---|---|
| | | or Slope- | Modulus - | or Slope- | Modulus - |
| | Caliper | CD | CD | MD | MD |
| Example | (mm) | (N/mm) | (N/mm²) | (N/mm) | (N/mm²) |
| Comparative Example: Polyethylene film with | 0.020 | 0.032 | 892.85 | 0.032 | 892.85 |
| basis weight of 17 gsm | | | | | |
| Sulflex white vegetable parchment paper with | 0.056 | 1.57 | 2744.34 | 3.20 | 5593.56 |
| basis weight of 50 gsm, available from Ahlstrom | | | | | |
| Bleached Kraft Machine Glazed Paper with a | 0.053 | 0.51 | 1047.46 | 0.88 | 1810.38 |
| basis weight of 43 gsm, available from Mondi | | | | | |
| Heerlen B.V. | | | | | |
| Inventive Example 1-C: PeltaMed 455 Bleached | 0.145 | 1.61 | 165.03 | 1.19 | 121.98 |
| Creped Waxed paper having a basis weight of | | | | | |
| 50 gsm, available from Pelta Medical Papers | | | | | |
| Inventive Example 2-C: PeltaMed Lightweight | 0.130 | 0.67 | 93.62 | 1.29 | 180.24 |
| Creped Waxed Paper having a basis weight of | | | | | |
| 48 gsm, available from Pelta Medical Papers | | | | | |
| Inventive Example 3-C: PeltaMed 455 | 0.180 | 0.88 | 46.06 | 1.07 | 56.32 |
| Bleached Creped Waxed Embossed paper | | | | | |
| having a basis weight of 50 gsm, available | | | | | |
| from Pelta Medical Papers | | | | | |

In additional to adequate fluid handling characteristics and being comfortable for wear, users also prefer products that are discrete. Generally, products that are noisy are not considered discrete. For example, as a user unwraps the liner and/or transports and applies the liner, the user prefers to avoid attracting any attention. Thus, to have a discrete product, the product should not be relatively noisy. It has been found that papers such as parchment papers and traditional bleached kraft machine glazed papers are 2 to 3 times more noisy than traditional plastic films used as barriers in absorbent articles. However, the creping process can transform a kraft machine glazed paper into a lower noise paper. Additional processes like embossing can reduce noise even further. In the case of paper materials, it is preferred that the paper decibel level is maintained below about 61 dB or, more preferably, below about 58 dB. Table 7 includes data for various paper products and their associated decibel (dB) level, as determined by the Sound test method disclosed herein.

TABLE 7

| | Average Loudness from |
|---|---|
| | 2,000 Hz-6,300 Hz |
| Example | Frequencies (dB) |
| Comparative Example: Always Ultra | 48.7 |
| Backsheet Film (polyethylene film) | |
| Sulflex white Vegetable Parchment | 73.8 |
| paper with basis weight of 50 gsm, | |
| available from Ahlstrom | |
| Bleached Kraft MG paper Uncoated | 59.1 |
| with a basis weight of 35 gsm | |
| Inventive Example 1-C: PeltaMed | 57.5 |
| 455 Bleached Creped Waxed paper | |
| having a basis weight of 50 gsm, | |
| available from Pelta Medical Papers | |
| Inventive Example 2-C: PeltaMed | 56.0 |
| Lightweight Creped Waxed Paper | |
| having a basis weight of 48 gsm, | |
| available from Pelta Medical Papers | |
| Inventive Example 3-C: PeltaMed | 51.3 |
| 455 Bleached Creped Waxed | |
| Embossed paper having a basis | |
| weight of 50 gsm, available | |
| from Pelta Medical Papers | |

As previously discussed, the first barrier layer and/or the second barrier layer are used to prevent fluid from leaking from the absorbent article. Generally, paper is known to absorb fluid and eventually lose strength when fluid is absorbed. It has been found that to have a robust first barrier layer and/or a second barrier layer that maintains sufficient strength during wear, application, and removal. It has been found that at least one of the first barrier layer and the second barrier layer has a wet burst strength of at least 10 kPa or at least about 12 kPa and/or a dry burst strength of at least about 80 kPa or at least about 100 kPa or at least about 200 kPa. The following data in Table 8 includes the dry burst strength as determined by standard test method ISO 2758:2014 and the wet burst strength as determined by standard test method ISO 3689. As demonstrated by the data in Table 8, Inventive Examples 1-C, 2-C, and 3-C each have the desired wet and dry burst strength. It is to be noted that the Sulflex white vegetable parchment paper has the desired wet and dry bust strength, but as described herein, the Sulflex white vegetable parchment paper exceeds the desired noise level and fails to be meet the requirements for flexibility, as evidenced by Table 6 and Table 7.

TABLE 8

| | Dry Burst | Wet Burst |
|---|---|---|
| | Strength | Strength |
| Example | (kPa) | (kPa) |
| Sulflex white vegetable parchment | 195.0 | 117.0 |
| paper with basis weight of 50 gsm, | | |
| available from Ahlstrom | | |
| Inventive Example 1-C: PeltaMed | 102.0 | 14.0 |
| 455 Bleached Creped Waxed paper | | |
| having a basis weight of 50 gsm, | | |
| available from Pelta Medical Papers | | |
| Inventive Example 2-C: PeltaMed | 180.0 | 24.0 |
| Lightweight Creped Waxed Paper | | |
| having a basis weight of 48 gsm, | | |
| available from Pelta Medical Papers | | |
| Inventive Example 3-C: PeltaMed | 87.0 | 13.5 |
| 455 Bleached Creped Waxed | | |
| Embossed paper having a basis | | |
| weight of 50 gsm, available from | | |
| Pelta Medical Papers | | |
| Peltamed Medicreped Creped Waxed | 142.0 | 10.0 |
| Paper with 9.6% crepe ratio and | | |
| having a basis weight of 50 gsm | | |

In addition to have adequate fluid handling properties, being discrete, and having sufficient strength, manufactures must be able to assemble the liner. Converting processes to manufacture liners and absorbent articles is typically done at high speeds to meet the demand for products. Converting process for traditional liners that include various layers of synthetic materials uses a relatively high amount of tension on the materials so that they can be processed quickly and progress through the various converting processes without wrinkling or proceeding along unintended web paths. Further, portions of the converting process may include areas where the web is unsupported, and the material needs to be about to withstand these unsupported areas. Unlike traditional synthetic materials, paper materials generally have very little stretch in the machine direction. This is problematic for converting processes which require tension in the web. Also, it is most economical if various types of materials, synthetic and non-synthetic materials, such as plastic films and paper, can be converted using similar or the same converting line and equipment. It has been found that paper may be modified to have a certain machine direction stretch so that it can be converted using similar processes as synthetic films. It has been found that creped papers can be converted because they have a lower machine direction (MD) web modulus and have the ability to stretch in the machine direction. To allow for a paper material to be converted, the MD web modulus of the material, which may be at least one of the first barrier layer and the second barrier layer, of less than about 550 N/cm at 1%-2% strain, measured according to the Web Modulus test method disclosed herein, and a stretch or MD strain at break of at least about 10% or at least about 12% or at least about 15%, measured according to the Stretch at Break test method disclosed herein. As demonstrated by Table 9, each of Inventive Examples 1-C, 2-C, and 3-C have the ability to be processed on high speed converting lines. These materials may be used for at least one of the first barrier layer and the second barrier layer, as previous described.

TABLE 9

| | 1-2% Web | Stretch/MD |
|---|---|---|
| | Modulus - MD | strain at |
| Example | (N/cm) | break (%) |
| Comparative Example: Polyethylene | 199.67 | 11.0 |
| film having a caliper of 20 microns | | |
| Bleached Kraft MG Paper having a | 3872.00 | 4.30 |
| basis weight of 43 gsm, available | | |
| from Mondi Herlene | | |
| Sulflex white vegetable parchment | 6357.00 | 0.90 |
| paper having a basis weight of | | |
| 50 gsm, available from Ahlstrom | | |
| Liner Brasil Industria E Comercia | 628.90 | 4.0 |
| Ltda 29 gsm Bleached Kraft paper | | |
| Peltamed Medicreped Creped Waxed | 1034.20 | 12.0 |
| Paper with 9.6% crepe ratio and | | |
| having a basis weight of 50 gsm | | |
| Inventive Example 1-C: PeltaMed | 400.55 | 18.0 |
| 455 Bleached Creped Waxed paper | | |
| having a basis weight of 50 gsm, | | |
| available from Pelta Medical Papers | | |
| Inventive Example 2-C: PeltaMed | 309.98 | 18.7 |
| Lightweight Creped Waxed Paper | | |
| having a basis weight of 48 gsm, | | |
| available from Pelta Medical Papers | | |
| Inventive Example 3-C: PeltaMed 455 | 324.50 | 19.7 |
| Bleached Creped Waxed Embossed | | |
| paper having a basis weight of 50 gsm, | | |
| available from Pelta Medical Papers | | |

### Test Methods

The following is a list of test methods for various parameters disclosed herein.

### Dunk Capacity

The dunk test determines the theoretical absorbent capacity of an absorbent article. Generally, the test sample is soaked in a test fluid for a prescribed amount of time, then subjected to a static weight on an inclined platform. Absorbent capacity is the amount of test fluid that is retained by the test sample. All testing is performed in a room controlled at 23° C.±3° C. and 50%±2% relative humidity and test samples are conditioned in this environment for at least 2 hours prior to testing.

The equipment needed to perform this test includes a liquid reservoir for soaking the test sample and an inclined platform and weight assembly for draining the excess test fluid. The liquid reservoir is a shallow dish that is large enough to allow the entire test sample to lie horizontally flat inside, and deep enough to keep the test sample fully submerged in the test fluid. The inclined platform is made of Plexiglass (or equivalent) and has a 15 degree angle with respect to the horizontal. The inclined platform has a length and width that is larger than that of the test sample and weight assembly. One of two different weight assemblies is used depending on the length of the test sample. For test samples with a longitudinal length less than or equal to 15.5 cm, the weight assembly has a mass of 1320 g±15 g with base dimensions of 15.5 cm by 5 cm. For test samples with a longitudinal length greater than 15.5 cm, the weight assembly has a mass of 2265 g±15 g with base dimensions of 20.5 cm by 6.5 cm. The weight assembly is foam-padded and constructed as follows. Lay a piece of polyethylene film (any convenient source) flat on a bench surface. A piece of polyurethane foam (25 mm thick; base dimensions determined by test sample length as previously stated; available from Concord-Renn Co. Cincinnati, OH, density of 1.0 lb/ft³, IDL 24 psi) is laid centered on top of the film. A piece of Plexiglas (6.4 mm thick; base dimensions determined by test sample length as previously stated) is then stacked on top of the polyurethane foam. Next the polyethylene film is used to wrap the polyurethane foam and Plexiglas plate securing it with transparent tape. A metal weight with handle is stacked on top of, and fastened to, the Plexiglass plate. In order to prevent the weight assembly from sliding down the inclined platform during the test, a support arm fixed to a ring stand with a non-slip base is used to keep the weight assembly in place.

The test fluid required for this method is prepared as follows using reagent grade components available from VWR International (or other equivalent source). To prepare 1000 grams of test fluid, begin by adding 800.00 g of deionized water to a 2000 mL beaker. Now add each of the following to the beaker: 20.00 g Urea (NH₂CONH₂, CAS 57-13-6), 9.00 g Sodium Chloride (NaCl, CAS 7647-14-5), 1.10 g Magnesium Sulfate Heptahydrate (MgSO₄× 7 H₂O, CAS 10034-99-8) and 0.60 g Calcium Chloride anhydrous (CaCl₂), CAS 10043-52-4). Now add an additional 169.30 g of deionized water. Transfer the beaker to a magnetic stir plate, add a magnetic stir bar to the beaker and mix thoroughly until all reagents have dissolved. The test fluid is stored and used at room temperature.

Test samples are prepared by removing the absorbent article from any outer packaging, and if the article is folded, unfold it. The protective layer (i.e., release cover) covering the attachment adhesive is left in place. Obtain the mass of the test sample (including protective layer covering the attachment adhesive) and record as Dry Mass to the nearest 0.01 g.

Fill the liquid reservoir with a sufficient amount of test fluid to keep the test sample submerged at least 5 mm beneath the surface of the test fluid for the entire soak time. With the topsheet side of the test sample facing the test fluid, submerge the test sample into the test fluid and allow it to soak for 25.0 minutes. After 25.0 minutes have elapsed, grip the test sample by one longitudinal edge, remove it from the test fluid and allow it to drip in a vertical position for 2.0 seconds. Using care not to bend or twist the test sample, transfer it to the inclined platform with the topsheet side facing the platform surface. While holding the test sample in place at one longitudinal end, gently place the weight assembly onto the test sample, ensuring that it is centered over the absorbent core of the test sample with the longitudinal axes of both aligned. Use the support arm to ensure the weight does not slide down the incline. The weight assembly is applied to the test sample for 2.0 minutes. After 2.0 minutes, gently remove the weight assembly. Grip the test sample by one longitudinal edge and allow it to drip in a vertical position for 2.0 seconds, once again ensuring the test sample is not bent or twisted in the process. Obtain the mass of the wetted test sample and record as Wet Mass to the nearest 0.01 g. Calculate the amount of liquid retained by the test sample by subtracting the Dry Mass from the Wet Mass, and record as Absorbent Capacity to the nearest 0.01 g.

In like fashion, repeat the entire procedure until a total of five replicate test samples are tested. Calculate the arithmetic mean for the absorbent capacity values measured for all five replicates and report as Absorbent Capacity to the nearest 0.01 g.

### Liquid Acquisition Time

Liquid Acquisition Times are measured for an absorbent article test sample insulted with a known volume of test fluid, using an apparatus described in detail in section 6 of compendial method NWSP 070.3.R0 (15). The test sample is subjected to a total of three consecutive liquid insults, and the time required for the test fluid from each liquid insult to pass into the test sample is recorded. All measurements are performed in a laboratory maintained at 23° C.±2° C. and 50%±2% relative humidity and test samples are conditioned in this environment for at least 2 hours prior to testing.

The test fluid required for this method is prepared as follows using reagent grade components available from VWR International (or other equivalent source). To prepare 1000 grams of test fluid, begin by adding 800.00 g of deionized water to a 2000 mL beaker. Now add each of the following to the beaker: 20.00 g Urea (NH₂CONH₂, CAS 57-13-6), 9.00 g Sodium Chloride (NaCl, CAS 7647-14-5), 1.10 g Magnesium Sulfate Heptahydrate (MgSO₄× 7 H₂O, CAS 10034-99-8), 0.60 g Calcium Chloride anhydrous (CaCl₂), CAS 10043-52-4), and 0.10 g Indigo Carmine blue dye (C₁₆H₈N₂Na₂O₈S₂, CAS 860-22-0). Now add an additional 169.20 g of deionized water. Transfer the beaker to a magnetic stir plate, add a magnetic stir bar to the beaker and mix thoroughly until all reagents have dissolved. The test fluid is used at room temperature.

The test apparatus includes a strikethrough plate, a baseplate, an electronic timer (accurate to 0.01 s), a funnel with a magnetic valve to discharge the dose of test fluid and a ring stand to hold the funnel, in accordance with the apparatus descriptions described in section 6 of compendial method NWSP 070.3.R0 (15). A suitable apparatus is the Lister AC available from Lenzing Instruments GmbH & Co (Gampern, Austria), or equivalent. In addition, a micropipette capable of delivering a 1.00 mL dose of test fluid is used.

Test samples are prepared by removing the absorbent article from any outer packaging, and if the article is folded, unfold it. The protective layer (i.e., release cover) covering the attachment adhesive is left in place. The test location is the intersection of the longitudinal and lateral midpoints of the absorbent article. Condition the test samples as previously described prior to testing.

Measure the liquid acquisition times as follows. The test sample is placed onto the baseplate, centering the test location over the plate. Place the strikethrough plate on top of the test sample with the test location centered below the center of the plate's orifice. Now place the entire stack of baseplate, test sample and strikethrough plate below the funnel such that the test location is centered below the funnel. Adjust the height of the funnel so that it is 30±1 mm above the top surface of the test sample (i.e., 5±1 mm above the top surface of the strikethrough plate. Connect the electrodes of the strikethrough plate to the electronic timer and ensure the timer is set to zero. With the discharge valve of the funnel closed, use a micropipette to dispense 1.00 mL of the test fluid into the funnel. Open the magnetic discharge valve of the funnel to discharge the 1.00 mL of test fluid into the reservoir of the strikethrough plate. The electronic timer will start as soon as the test fluid makes contact with the electrodes and will stop once the test fluid falls below the level of the electrodes (i.e., the entire liquid dose has penetrated into the test sample). Record the time indicated on the electronic timer as the first acquisition time to the nearest 0.01 seconds. Prior to testing the next sample, the electrodes and strikethrough plate are thoroughly rinsed with deionized water and dried completely.

In like fashion, the entire test sequence is repeated until a total of five replicate test samples have been tested. Calculate the arithmetic mean for the first acquisition time values recorded for all five replicate test samples and report as First Liquid Acquisition Time to the nearest 0.01 seconds. In like fashion, calculate the arithmetic mean for the second and third acquisition times and report as Second Liquid Acquisition Time and Third Liquid Acquisition Time, respectively, to the nearest 0.01 seconds.

### Standard Rewet

This rewet method measures the amount of fluid that emerges through the topsheet of an absorbent article test sample 20 minutes after the test sample has been insulted with a specified volume of test fluid. All testing is performed in a room controlled at 23° C.±3° C. and 50%±2% relative humidity and absorbent article test are conditioned in this environment for at least 2 hours prior to testing.

The rewet weight is constructed of stainless steel, or equivalent, such that the dimensions of the bottom face of the weight are 4.5 cm by 10 cm and the total mass of the weight is 3150 g, including any handle that might be attached. The total mass of the weight applies a pressure of 1 psi across the bottom face of the weight. A mechanical device can be constructed, if needed, to aid in the lowering and raising of the rewet weight.

The rewet substrate used for this test is two layers of filter paper with a diameter of 150 mm. A suitable filter paper is VWR qualitative filter paper Grade 413, which has a basis weight of about 73 gsm, a thickness of about 160 microns with medium porosity, and is available from VWR International (VWR European Cat #516-0817 as of 2023), or equivalent. The filter paper is conditioned at 23° C.±2° C. and 50%±2% relative humidity for at least 1 day (24 hours) prior to being used for testing. For each test sample replicate, a fresh stack of two layers of filter is required.

The test fluid required for this method is prepared as follows using reagent grade components available from VWR International (or other equivalent source). To prepare 1000 grams of test fluid, begin by adding 800.00 g of deionized water to a 2000 mL beaker. Now add each of the following to the beaker: 20.00 g Urea (NH₂CONH₂, CAS 57-13-6), 9.00 g Sodium Chloride (NaCl, CAS 7647-14-5), 1.10 g Magnesium Sulfate Heptahydrate (MgSO₄× 7 H₂O, CAS 10034-99-8), 0.60 g Calcium Chloride anhydrous (CaCl₂), CAS 10043-52-4) and 0.10 g Indigo Carmine blue dye (C₁₆H₈N₂Na₂O₈S₂, CAS 860-22-0). Now add an additional 169.20 g of deionized water. Transfer the beaker to a magnetic stir plate, add a magnetic stir bar to the beaker and mix thoroughly until all reagents have dissolved. The test fluid is stored and used at room temperature.

Test samples are prepared by removing the absorbent article from any outer packaging, and if the article is folded, unfold it. The protective layer (i.e., release cover) covering the attachment adhesive is left in place. The test location is an area that is 6 cm long (parallel to the longitudinal axis of the test sample) by 2 cm wide (perpendicular to the longitudinal axis of the test sample), centered over the intersection of the longitudinal and lateral midpoints of the absorbent article test sample. Mark the test location on the body-facing side of the test sample using a permanent ink pen with a fine tip. In like fashion, prepare a total of five replicate test samples. Condition the test samples as previously described prior to testing.

Execute the rewet test as follows. Place the absorbent article test sample onto a rigid, horizontal surface with the body-side of the article facing up. Add 1 mL of test fluid to a volumetric pipette and position the pipette over the center of the previously marked test location such that the tip of the pipette is about 10 mm above the surface of the test sample. Use a stopwatch to dispense the test fluid in a dropwise manner to evenly distribute the test fluid across the entire test location in 30 seconds. As soon as the test fluid has been fully dispensed to the test sample, start a 20 minute timer. Measure the mass of two layers of filter paper and record as Initial Mass to the nearest 0.001 g. Arrange the layers of pre-weighed filter paper such that each layer is centered upon the next to form a neat stack. After 20 minutes have elapsed, place the stack of filter paper onto the body-facing side of the test sample, centering it over the test location. Now gently lower the rewet weight onto the filter paper such that the weight is centered over the filter paper and test sample. As soon as the rewet weight is in place, start a 15 second timer. After 15 seconds have elapsed, remove the rewet weight. Now measure the mass of the stack of filter paper and record as Final Mass to the nearest 0.001 g. Subtract the Initial Mass from the Final Mass and record as 20 Minute Rewet to the nearest 0.001 g. Wipe off any residual test fluid from the bottom face of the rewet weight prior to testing the next sample.

In like fashion, repeat the entire procedure until all five replicate test samples have been tested. Calculate the arithmetic mean of the 20 minute rewet values for all five replicates, and report as Standard Rewet to the nearest 0.001 grams.

### Capillary Flow Porometry

Capillary Flow Porometry (CFP), also known as gas-liquid porometry, is used to determine the first bubble point (FBP; largest pore diameter), mean flow pore diameter and smallest pore diameter of a porous structure by analyzing both the wet and dry test specimen. CFP provides pore size distribution data as well. Using a pressure scan technique in which the pressure is increased linearly over time, data sampling of the applied pressure and corresponding flow rate is performed continuously which enables the calculation of the aforementioned parameters. All testing is performed in a room controlled at 23° C.±3° C. and 50%±2% relative humidity and test specimens are conditioned in this same environment for at least 2 hours prior to testing.

CFP is a measurement technique based on the displacement of an inert wetting liquid from the pores of a test specimen by applying test gas at increasing pressure. By measuring the pressure at which a liquid is pressed out of the pores of a test specimen, the diameter of the pores can be calculated using the Young-Laplace equation, ΔP=4γ*cos θ/D, where ΔP is differential pressure, γ is the surface tension of the wetting liquid, θ is the contact angle between the liquid and the capillary wall and D is the diameter of the pore. The tortuosity or symmetry of the pores within the test specimen is unknown, therefore an assumption is made that no tortuosity is present. Thus, all capillaries are assumed to be perfectly cylindrical, and no shape correction factor is used for the calculations herein. Wetting liquids have very low surface tension which results in a contact angle of 0°, thus cos θ=1. The wetting liquid used for this method is Galwick®, available from Porous Materials Inc., Ithaca, NY, or equivalent, having a surface tension of 15.9 mN/m. Galwick® is a perfluorinated liquid that has been oxidized and polymerized (1,1,2,3,3,3-Hexafluoro-1-propene; CAS 69991-67-9). An alternate wetting liquid with the same surface tension can be used in place of the Galwick® fluid.

The test begins with a fully wetted test specimen in the sample chamber. To generate the wet curve, the instrument builds up pressure inside the pressure chamber starting from ambient pressure, over the wetted specimen. Gradually the pores in the test specimen will start to open as the wetting fluid is displaced beginning with the largest pore, and a gas flow rate is measured by the flow meter. The pressure build up is performed until all pores in the test specimen are opened and the specimen has completely dried. The instrument then deflates, and a second run is performed to generate the dry curve.

Measurements are made on test specimens taken from rolls or sheets of the raw material. Alternatively, test specimens are obtained from a material layer removed from an absorbent article. When excising the material layer from an absorbent article, care is used to prevent any contamination or distortion to the layer during the process. The excised layer should be substantially free from residual adhesive. The test specimen is obtained from a region of the material layer that is free from folds or wrinkles, and it is cut to a diameter of 25 mm using a cutting die or other sharp blade. A total of five replicate test specimens are prepared Test specimens are to be handled along the very outermost edges only using laboratory tweezers so as to prevent any contamination to the test surfaces of the specimen.

Apparatus suitable for the porometry measurements is a low pressure (up to 1.5 bar) gas-liquid displacement porometer that enables a measurable pore size range from 0.424-500 microns when using a wetting liquid with a surface tension of 15.9 mN/m. The porometer consists of a pressure sensor (at least 2 bar), a pressure regulator, two flow meters for measuring low (up to 10 L/min) and high (up to 200 L/min) flow rates with auto-switching between meters, a flow sensor, a sample holder (for 25 mm diameter test specimens) and an operating panel. The sample holder consists of several elements including the following, listed in order from bottom to top: a sample holder base, a primary support grid, a secondary support grid on which the test specimen is placed, a scaling ring (O-ring that creates an effective specimen diameter of 18.5 mm) and a sample holder cap that is connected to the pressure tube. The support grids allow free passage of the test gas but are adequately rigid to prevent deformation of the test specimen throughout the test. One means of providing adequate support for the test specimen is to use a primary support grid with a thickness of about 0.86 mm that has 1.5 mm² diamond-like shape openings with a total open area of about 33%, and a secondary support grid made of 20 mesh woven wire with a thickness of about 0.64 mm. A compressed air supply (dry, clean, oil-free) between 5-7 bar is required for the control air and a separate compressed air supply (dry, clean, oil-free) between 6-16 bar is required as the test medium. A porometer suitable for this method is the Porolux™ 100NW (available from IB-FT GmbH, Berlin, Germany), or equivalent, interfaced to a computer running complimentary software such as LabVIEW, and capable of continuously collecting pressure, flow rate, pore diameter, and cumulative filter flow (percent filter flow) throughout the test.

The porometry test is executed as follows. The regulator for the test pressure is adjusted to reach a reading of 2 bar on the manometer of the instrument. The regulator for the control air is set to about 7 bar. Soak the test specimen in the wetting liquid for about 5 minutes. Using laboratory tweezers, grasp the edge of the test specimen and transfer it to the secondary support grid inside the sample holder. Ensure that the specimen is centered inside the sample chamber and that no wrinkles are present. Insert the scaling ring over the test specimen and secure the sample holder cap to seal the test specimen inside the chamber. Set the porometer to run in full porometry mode where the wet curve is generated, followed by the dry curve. For the initial test run, the pressure is set to ramp up from 0 bar until it reaches 1.5 bar over the course of 100 steps. Start the test run and continuously collect flow rate (L/min) and pressure (bar) data for the wet and dry test specimen. The accuracy of the measurement directly depends on the number of steps (i.e., data points). Thus, the number of steps entered and the final pressure setting are adjusted, as needed, after the initial test run is complete to balance the needed accuracy of the test. One of skill will understand the need to adjust the final pressure if, for example, the maximum flow rate is reached well before the selected final pressure. One of skill will also understand that for hard to dry specimens, the dry curve is to be generated prior to the wet curve. At the end of the test, the test specimen is removed from the sample chamber and discarded.

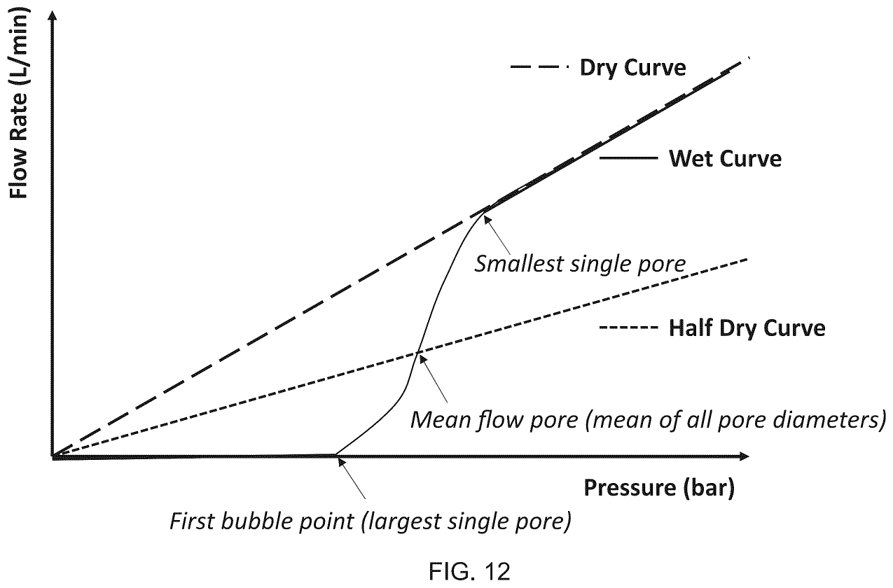

A graph of flow rate (L/min) versus pressure (bar) is constructed and the following three curves are plotted: a wet curve, a dry curve and a curve that represents the “half dry” curve. The half dry curve is generated by dividing the flow values of the dry curve by 2 across the array of corresponding pressures. The point on the graph where the dry curve first coincides with the wet curve corresponds to the pressure value at which the flow rates for the wet and dry test specimen become the same because at this point, all of the pores have been emptied. Determine the pressure value at the point where the dry curve first coincides with the wet curve and record as smallest pore pressure to the nearest 0.0001 bar. The point on the graph where 50% of the total gas flow can be accounted for occurs where the wet curve intersects the half dry curve. Determine the pressure value at the point where the wet curve intersects the half dry curve and record as mean flow pore pressure to the nearest 0.0001 bar. The pressure at which the first flow of test gas occurs through the test specimen is known as the first bubble point. Determine the pressure value at the first instance where there is a detectable gas flow on the wet curve and record as largest pore pressure to the nearest 0.0001 bar. FIG. 12 illustrates an example graph that depicts the three curves with the points of interest marked.

Calculate the pore diameter that corresponds to each of the recorded pressure values using the Young-Laplace equation as follows:

$$D = [ 4 γ * cos θ / Δ P ] * {10}^{8}$$

where ΔP is the differential pressure value recorded in bar
γ is the surface tension of the wetting liquid (15.9 mN/m for Galwick®)
cos θ is 1
D is the pore diameter in microns

For the largest pore pressure, the calculated pore diameter is recorded as Largest Pore Diameter to the nearest 0.01 micron. For the smallest pore pressure, the calculated pore diameter is recorded as Smallest Pore Diameter to the nearest 0.01 micron. For the mean flow pore pressure, the calculated pore diameter is recorded as Mean Flow Pore Diameter to the nearest 0.01 micron.

In the instance that a given test specimen has zero flow at even the highest applied pressure, and thus none of the three curves can be plotted, it is assumed that all of the pores within that test specimen are less than the smallest measurable pore. When using a wetting liquid with a surface tension of 15.9 mN/m and a final pressure of 1.5 bar, the smallest measurable pore is 0.424 micron.

A graph of cumulative filter flow (percent flow) versus diameter (micron) is constructed. From this plot, the pore size distribution of the test specimen is obtained. For example, this plot depicts which percentage of flow (at the y-axis) has passed through the pores with a diameter larger than the value at the corresponding point at the x-axis. Using the plot, determine the diameter value that corresponds to 10% flow and record this value as 10% of Pore to the nearest 0.01 micron. At the 10% of Pore value, 90% of the pores in the test specimen are smaller than this value.

In like fashion, repeat the entire procedure until all five replicate test specimens have been tested. Calculate the arithmetic mean of all five replicates for each of the calculated parameters and report as Largest Pore Diameter, Smallest Pore Diameter, Mean Pore Diameter, and 10% of Pore, all to the nearest 0.01 micron.

### Water Repellency

The water repellency method measures the time it takes water to penetrate the structure of a test specimen, and generally follows the procedure described in European Standard EN 868-3:2017, with modifications specified herein. Penetration time is determined by sprinkling a water activated ultraviolet (UV) indicator powder on the upper surface of a dry test specimen, then floating the test specimen on the surface of water in a reservoir placed under controlled UV lighting. The specimen is closely monitored while measuring the time it takes for general fluorescence to occur, indicating that water has permeated the structure of the specimen. All testing is performed in a room controlled at 23° C.±3° C. and 50%±2% relative humidity and test specimens are conditioned in this same environment for at least 2 hours prior to testing.

The apparatus required for this test include a UV light source, a shallow dish (at least 80 mm by 80 mm with a depth of at least 15 mm) and a stopwatch. A UV light source that has a wavelength range of 315 nm to 390 nm is required to detect fluorescence. An example of a suitable UV light source is a portable, hand-held UV-A (365 nm) lamp such as model ENF-280C available from Spectro-UV, Farmingdale, NY, or equivalent. The shallow dish is filled with room temperature deionized water (23° C.±1° C.) to a depth of about 10 mm. The UV light source is mounted directly above the dish of water such that the light panel is about 150 mm above the surface of the water.

The UV indicator powder is prepared as follows. Finely ground sucrose (for example, 10× confectioner's sugar; any convenient source) is used as an inactive ingredient that serves as a general purpose powder filler for the fluorescent powder. The fluorescent powder is Sodium Fluorescein (CAS 518-47-8, reagent grade, available from any convenient source). The sucrose is dried overnight in an oven set to 110° C., and then passed through a 200 mesh sieve. Place about 20 g of the dried, sieved sucrose and about 0.1 g of sodium fluorescein into a mortar and pestle and thoroughly blend the powder mixture. Transfer the prepared indicator powder to a dispensing bottle fitted with a 100 mesh screen as the lid. Store the bottle of indicator powder in a dessicator to prevent moisture from causing clumps in the powder.

Measurements are made on test specimens taken from rolls or sheets of the raw material. Alternatively, test specimens are obtained from a material layer removed from an absorbent article. When excising the material layer from an absorbent article, care is used to prevent any contamination or distortion to the layer during the process. The excised layer should be substantially free from residual adhesive. The test specimen is obtained from a region of the material layer that is free from folds or wrinkles, and it is cut to a 60 mm by 60 mm square using a cutting die or other sharp blade. Test specimens are to be handled only along the very outermost edges using laboratory tweezers so as to prevent any contamination to the test surfaces of the specimen. A support frame is used to prevent the test specimen from curling during the test and also to prevent erroneous wetting effects that can occur along the cut edge of the test specimen. The support frame is prepared from heavyweight transparency film (˜134 g/m²; any convenient source) as follows. Cut a sheet of transparency film into a 70 mm by 70 mm square and then cut out a 58 mm by 58 mm square window from the center such that the sides of the outer and inner squares are parallel to one another, forming a clear plastic frame. Place the prepared support frame onto a rigid horizontal surface and then center the test specimen over the cut out window such that the test side of the test specimen is facing down. Secure the test specimen to the frame along all four edges using a general purpose clear tape. The tape overlaps the edges of the test specimen by no more than 2 mm, and no portion of the tape extends into the window portion of the support frame. Trim away any excess tape that extends beyond the outermost edges of the support frame using a sharp blade. In like fashion, a total of ten replicate test specimens are prepared and secured to individual support frames, with five replicates having a first side facing down inside the support frame and the other five replicates having a second side facing down inside the support frame.

The test is executed as follows. The UV light source is turned on and allowed to come to full power. Ensure the shallow dish that is positioned below the UV light source is filled to a depth of about 10 mm with room temperature deionized water. The prepared test specimen in its support frame is placed onto a rigid horizontal surface with the first test side facing down. The indicator powder is applied to the non-test side of the specimen by sprinkling it from the dispenser bottle until the entire surface of the test specimen is uniformly coated. The coated test specimen is transferred to and positioned above the surface of the water in the shallow dish, ensuring that the layer of indicator powder on the non-test side of the specimen is not disturbed. The test side of the test specimen is gently floated onto the water surface, and the stopwatch is started as soon as the test specimen contacts the water. The non-test side of the specimen is closely observed until the first appearance of fluorescence occurs. The stopwatch is stopped, and the time is recorded as water repellency time to the nearest 1 second, noting the test side as either first or second side. The test specimen is removed from the shallow dish of water and discarded. The water in the dish is properly discarded and the dish is thoroughly rinsed with clean deionized water, then dried and returned to its position beneath the UV light source. In like fashion, the procedure is repeated until all five replicates of the first test side and all five replicates of the second test side have been tested.

The arithmetic mean for the ten time values recorded for the ten test replicates is reported as Repellency Time to the nearest 1 second.

### Wet Through

The wet through method measures the amount of test fluid that passes through the back side of an absorbent article test sample or, stated another way, the side of the absorbent article test sample that is intended to attach to the wearer's undergarments (a garment-facing side) after it has been loaded with a specified volume of test fluid and then subjected to a specified pressure for 10 minutes. All testing is performed in a room controlled at 23° C.±3° C. and 50%±2% relative humidity and absorbent article test samples as well as the standard cotton fabric used in this method are conditioned in this environment for at least 2 hours prior to testing.

For the wet through method, a pressure of 19 g/cm² (0.27 psi) is applied over the full absorbent article test sample using a weight assembly prepared as follows. A plexiglass plate having a thickness of at least 6 mm is cut such that the length and width are equivalent to the length and the width (at widest region) of the full test sample. Weights are then added to the plexiglass plate such that the total mass of the weights and the plate impart a pressure of 19 g/cm².

To determine the amount of test fluid that passes through the backsheet of the absorbent article test sample, a stack of pre-weighed standard cotton fabric is placed beneath the absorbent article for the duration of the test, and then weighed again after the test is over. The standard cotton fabric used for this method is white 100% cotton weave with a basis weight of about 100 g/m² (style #429-W) available from Testfabrics, Inc, West Pittston, Pennsylvania, USA, or equivalent. The standard cotton material is cut to a length that is about 3 cm longer than the length of the absorbent article (measured from the front edge to rear edge) and a width that is at least as wide as the widest portion of the absorbent article test sample. For each test replicate, a fresh stack of three layers of the standard cotton fabric is required.

The test fluid required for this method is prepared as follows using reagent grade components available from VWR International (or other equivalent source). To prepare 1000 grams of test fluid, begin by adding 800.00 g of deionized water to a 2000 mL beaker. Now add each of the following to the beaker: 20.00 g Urea (NH₂CONH₂, CAS 57-13-6), 9.00 g Sodium Chloride (NaCl, CAS 7647-14-5), 1.10 g Magnesium Sulfate Heptahydrate (MgSO₄× 7 H₂O, CAS 10034-99-8), 0.60 g Calcium Chloride anhydrous (CaCl₂), CAS 10043-52-4) and 0.10 g Indigo Carmine blue dye (C₁₆H₃N₂Na₂O₈S₂, CAS 860-22-0) and 10 g of Pegosperse® surfactant (Polyethylene Glycol Monolaurate, CAS 9004-81-3). Now add an additional 159.20 g of deionized water. Transfer the beaker to a magnetic stir plate, add a magnetic stir bar to the beaker and mix thoroughly until all reagents have dissolved. The test fluid is used at room temperature.

Test samples are prepared by removing the absorbent article from any outer packaging, and if the article is folded, unfold it. The protective layer (i.e., release cover) covering the attachment adhesive is left in place, for now. In like fashion, prepare a total of five replicate test samples.

Execute the wet through test as follows. Record the mass of three layers of standard cotton fabric as Initial Mass to the nearest 0.001 g. Arrange the layers of pre-weighed cotton fabric such that each layer is centered upon the next to form a neat stack, and place the stack onto a rigid, horizontal surface. Now remove the protective layer (i.e., release cover) that covers the attachment adhesive from the absorbent article test sample. Place the adhesive side of the test sample onto the stack of cotton fabric with the body-facing side of the test sample facing up, ensuring that the test sample is centered over the cotton fabric stack. Add 2 mL of test fluid to a volumetric pipette and position the pipette over the center of the test sample (intersection of the longitudinal and lateral midpoints) such that the tip of the pipette is about 10 mm above the surface of the test sample. Use a stopwatch to dispense the test fluid to the test sample continuously over 30 seconds. As soon as the test fluid has been fully dispensed to the test sample, start a 10 minute timer. When 10 minutes have elapsed, place the weight assembly onto the test sample such that the plexiglass plate is centered over the absorbent core of the test sample and the additional weights are placed on top of the plexiglass plate. When the weight assembly is in place, immediately start a 10 minute timer. After 10 minutes have elapsed, remove the weight assembly from the test sample, then remove the test sample from the stack of cotton fabric and discard the test sample. Now record the mass of the stack of cotton fabric as Final Mass to the nearest 0.001 g. Subtract the Initial Mass from the Final Mass and record as Wet Through to the nearest 0.001 g.

In like fashion, repeat the entire procedure until all five replicate test samples have been tested, using a fresh stack of standard cotton fabric for each replicate. Calculate the arithmetic mean for the wet through values measured for all five replicates and report as Wet Through to the nearest 0.001 g.

### Sound

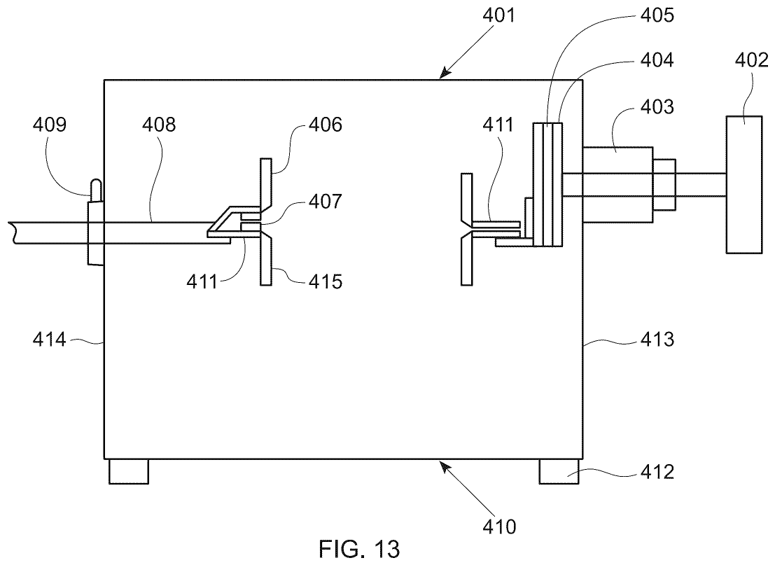

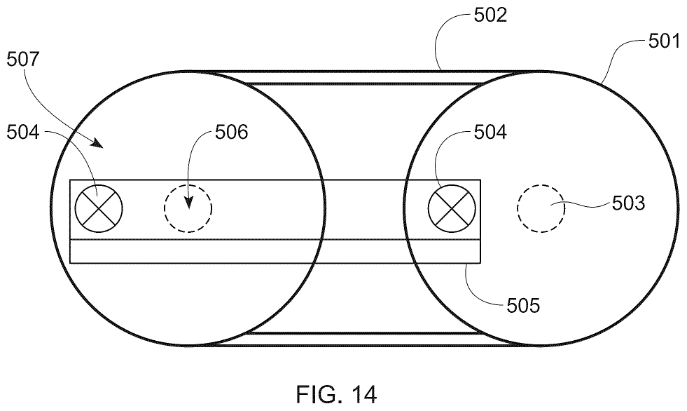

The test sample is tested using a testing mechanism comprised of a box (shown in FIG. 13) having internal dimensions 192 mm wide by 203 mm deep by 198 mm in height. The box is made out of a rigid material able to hold the weight of the rotational mechanism (shown in FIG. 14) and stand the forces inflicted during testing without deformation. One such suitable material is Lexan with a thickness of 12 mm. The box has an opening at the top surface 401 large enough to allow mounting film samples on the test brackets 411 and allow access to the rotational mechanism for adjustments as needed. The box is mounted on rubber footings 412, one on each corner of the bottom surface of the box. The bottom of the box 410 is closed with a rigid material to provide stability to the box.

A block with dimensions 13.1 cm long by 2.54 cm deep by 4 cm tall serves at the supporting base 403 of the rotational mechanism and it is attached to the front surface of the box 413. Base 403 is made of Nylon or other similar material. Base 403 supports two pins 503 and 506 on which two nylon pulleys 501 and 507 are free to rotate. The nylon pulleys are 7.5 cm in diameter and are connected by a rubber belt 502 in a way that both pulleys rotate at the same speed when the mechanism is activated. On the opposite end of pin 506, a wheel 402 is attached which is used to operate the mechanism. A side view of the pulley 404 and the belt 405 are shown in FIG. 13 as they are located in the box.

A mounting bracket 505 is attached to both pulleys at attachment point 504. Point 504 is mounted on the pulley at a radial distance of 25 mm from the pin 503 on pulley 501 and at a radial distance of 25 mm from the pin 506 on pulley 507. Attached to the mounting bracket 505 is clamp 411. Clamp 411 is at least 100 mm wide and has rubber pads 407 at the top and bottom of the clamp to properly secure the test sample.

A second clamp identical to clamp 411 is mounted on a rod 408 which is located opposite to the first clamp. The rod 408 goes thru a hole on the back surface of the box 414. The hole contains a locking mechanism 409 which when disengaged allows the clamp 411 to slide towards the back of the box 414 for sample loading and slide towards the clamp mounted to the rotational mechanism for sample testing.

Each clamp 411 has a piece of Lexan 406 with dimensions 100 mm long, 32 mm tall and 1.5 mm thick attached to the top jaw of the clamp. A second piece of Lexan 415 with dimensions 100 mm long, 54 mm tall and 1.5 mm thick is attached to the bottom jaw of the clamp. The means used to affix the plexiglass to the clamp will vary depending on the clamp design selected and are not intended to be prescribed. The attachments must be able to stand the stresses induced during testing.

When the clamp 411 on mounting bracket 505 is moved to its highest vertical position, it will be parallel with clamp 411 mounted on rod 408, but at a vertical distance of 10 mm. Test samples loaded in this position will maintain both ends of the test specimen parallel with each other.

When the mechanism is activated the first clamp 411 on mounting bracket 505 will rotate in a circular path with a 23 mm radius while the second clamp 411 on rod 408 stays stationary. To load a film test specimen of 100 mm×100 mm unlock the locking mechanism 409 to allow the rod 408 to move and set the distance between the first and second clamps 411 to 67 mm. The first clamp on mounting bracket 505 is moved to its highest vertical position and the film test specimen is clamped with one edge in the first clamp and the opposite edge in the second clamp. Once the test specimen is secured in the clamps unlock the locking mechanism 409 and slide back the second clamp 411 mounted on rod 408 and set the distance between clamps to 15 mm. Lock the locking mechanism to prevent the second clamp from moving back.

The box 401 is placed inside an Audiometric test enclosure with a Noise Isolation Class (NIC) equal or higher to 38 as calculated in accordance with Classification ASTM E 413 Test Method E596. The microphone with preamplifier of the sound meter is placed in the test enclosure 50 mm above the second stationary clamp 411 and centered over the test sample. The analyst performing the test steps into the audiometric test chamber with the sound meter and closes the door of the chamber before setting the sound meter instrument to collect data. The analyst turns the wheel of the testing mechanism (402) at a rate of 1 revolution per second for 30 seconds and the sound pressure level is recorded by the sound meter at the ⅓ octave frequency range between 2000 Hz and 6300 Hz. Afterwards the analyst stops the sound meter data collection and steps out the Audiometric test enclosure. The data from the sound meter is later downloaded to a computer for analysis. It is recommended to select an integrating sound meter which meets the following standards: IEC 61672-1:2002, ANSI S1.4, ANSI S1.4. The sound meter must have data logging software of spectral data and ⅓ octave band frequency analysis.

### Three Point Bend

The bending properties of a test sample are measured using an ultra sensitive three point bend test on a universal constant rate of extension test frame (a suitable instrument is the MTS Alliance using TestSuite Software, as available from MTS Systems Corp., Eden Prairie, MN, or equivalent) equipped with a load cell appropriate for the forces being measured. The test is executed on test specimens prepared for both MD (machine direction; parallel to the longitudinal axis of the absorbent article) and CD (cross direction; perpendicular to the longitudinal axis of the absorbent article) bending. All testing is performed in a room controlled at 23° C.±3° C. and 50%±2% relative humidity.

The ultra sensitive three point bend method is designed to maximize the force signal to noise ratio when testing materials with very low bending forces. The force signal is maximized by using a high sensitivity load cell (e.g., 5N), using a small span (load is proportional to the span cubed) and using a wide specimen width (total measured load is directly proportional to width). The fixture is designed such that the bending measurement is performed in tension, allowing the fixture mass to be kept to a minimum. Noise in the force signal is minimized by holding the load cell stationary to reduce mechanical vibration and inertial effect and by making the mass of the fixture attached to the load cell as low as possible.

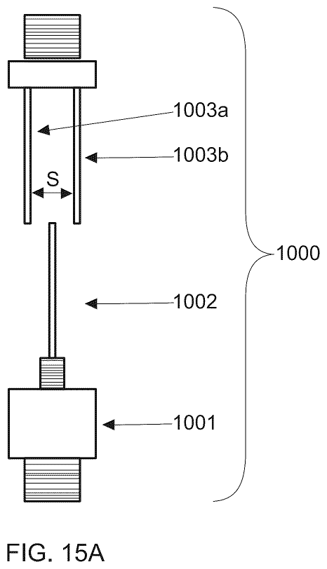

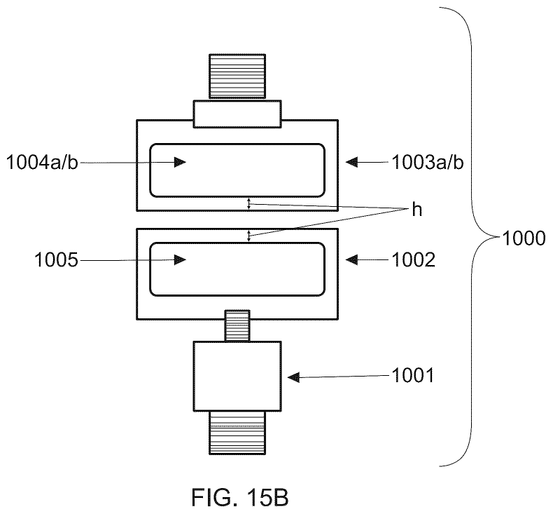

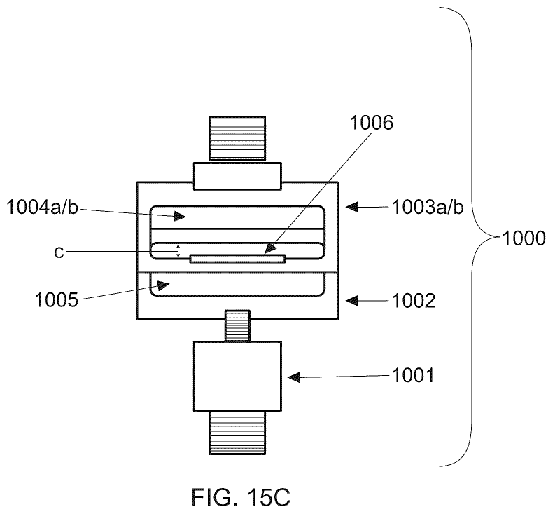

Referring to FIGS. 15a-15c, the load cell 1001 is mounted on the stationary crosshead of the universal test frame. The ultra sensitive fixture 1000 consists of three thin blades constructed of a lightweight, rigid material (such as aluminum, or equivalent). Each blade has a thickness of 1.0 mm, rounded edges and a length that is able to accommodate a bending width of 100 mm. Each of the blades has a cavity 1004a and 1004b (outside blades) and 1005 (central blade) cut out to create a height, h, of 5 mm of blade material along their horizontal edges. The two outside blades 1003a and 1003b are mounted horizontally to the moveable crosshead of the universal test frame, aligned parallel to each other, with their horizontal edges vertically aligned. The span, s, between the two outside blades 1003a and 1003b is 5 mm+0.1 mm (inside edge to inside edge). The central blade 1002 is mounted to the load cell on the stationary crosshead of the universal test frame. When in place, the central blade 1002 is parallel to the two outside blades 1003a and 1003b and centered at the midpoint between the outside blades 1003a and 1003b. The blade fixtures include integral adapters appropriate to fit the respective positions on the universal test frame and lock into position such that the horizontal edges of the blades are orthogonal to the motion of the crossbeam of the universal test frame.

Absorbent article samples are conditioned at 23° C.±3° C. and 50%±2% relative humidity two hours prior to testing. Test specimens are taken from an area of the sample that is free from any seams and residua of folds or wrinkles. The specimens are prepared by cutting them to dimensions of 100 mm, which is the sample width, by 50 mm, which is the sample length. For MD bending (i.e., bending normal to the longitudinal axis of the article), the long side of the test specimen is parallel to the longitudinal axis of the article. For CD bending (i.e., bending normal to the lateral axis of the article), the long side of specimen is parallel to the lateral axis of the article. The side of the test specimen that faces the surface of the absorbent article (or the side intended to face the surface of a finished article) is marked and the orientation (i.e., MD and CD) is maintained after the specimens are cut. In like fashion, five replicate test specimens are prepared for MD bending and five separate test specimens are prepared for CD bending.

The universal test frame is programmed such that the moveable crosshead is set to move in a direction opposite of the stationary crosshead at a rate of 1.0 mm/s. Crosshead movement begins with the specimen 1006 lying flat and undeflected on the outer blades 1003a and 1003b, continues with the inner horizontal edge of cavity 1005 in the central blade 1002 coming into contact with the surface of the specimen 1006, and further continues for an additional 7 mm of crosshead movement, and then stops to end the test. The crosshead then returns to zero. Force (N) and displacement (mm) are collected at 50 Hz throughout.

Prior to loading the test specimen 1006, the outside blades 1003a and 1003b are moved towards and then past central blade 1002 until there is approximately a 3 mm clearance, c, between the inner horizontal edges of cavities 1004a and 1004b in the outside blades 1003a and 1003b and the inner horizontal edge of cavity 1005 in the central blade 1002 (see FIG. 15c). The specimen 1006 is placed within clearance c such that it spans the inner horizontal edges of cavities 1004a and 1004b in the outside blades 1003a and 1003b. For MD bending, the test specimen is oriented such that the CD (short side) of the specimen is perpendicular to the horizontal edges of the blades, and the article-facing surface of the specimen is facing up. For CD bending, the test specimen is oriented such that the MD (short side) of the specimen is perpendicular to the horizontal edges of the blades and the article-facing surface of the specimen is facing up. Center the specimen 1006 between the outside blades 1003a and 1003b. Slowly move the outside blades 1003a and 1003b in a direction opposite of the stationary crosshead until the inner horizontal edge of cavity 1005 in the central blade 1002 touches the top surface of the specimen 1006. Start the test and continuously collect force and displacement data.

Force (N) is plotted versus displacement (mm). The maximum peak force is recorded to the nearest 0.001 N. The slope of the linear portion of the force versus displacement curve is determined and recorded as slope to the nearest 0.001 N/mm. The area under the curve from load onset up to the maximum peak force is calculated and recorded as energy to peak to the nearest 0.001 N-mm. In like fashion, repeat the entire test sequence for a total of five MD bending test specimens and five CD bending test specimens.

For each test specimen type (MD and CD), the arithmetic mean of the maximum peak force among like specimens is calculated to the nearest 0.001 N and recorded as MD Peak Load and CD Peak load, respectively. For each test specimen type (MD and CD), the arithmetic mean of the slope among like specimens is calculated to the nearest 0.001 N/mm and reported as MD Slope and CD Slope, respectively. For each test specimen type (MD and CD), the arithmetic mean of the energy to peak among like specimens is calculated to the nearest 0.001 N-mm and reported as MD Energy to Peak and CD Energy to Peak, respectively.

Stiffness-CD is equal to CD Slope, Stiffness-MD is equal to the MD Slope.

Flexural Modulus is calculated as follows:

$$Flexural modulus = ( Slope * Span \hat{} 3 ) / ( 4 * Sample Width * Caliper \hat{} 3 )$$

### Where:

Slope is the MD Slope or CD slope of the respective direction where Flexural modulus is being reported. Span, s, is the between the two outside blades, as described above. Caliper is the caliper of the test sample and is determined by the Caliper (Raw Material) method.

### Web Modulus and Stretch at Break

The tensile properties (tensile strength, stretch, and energy absorption) of a test sample are calculated from measured force and elongation values obtained using a constant rate of elongation until the sample breaks. The test is run in accordance with compendial method ISO 1924-3, with modifications noted herein. Measurements are made on a constant rate of extension tensile tester using a load cell for which the forces measured are within 1% to 99% of the limit of the cell. A suitable instrument is the MTS Alliance using Test Suite Software, available from MTS Systems Corp., Eden Prairie, Minn., or equivalent. All measurements are performed in a laboratory maintained at 23° C.±2° C. and 50%±2% relative humidity and test samples are conditioned in this environment for at least 2 hours prior to testing.

Measurements are made on both MD (machine direction) and CD (cross direction) test samples taken from rolls or sheets of the raw material, or test samples obtained from a finished package. When excising the test sample from a finished package, use care to not impart any contamination or distortion to the sample during the process. The excised sample should be free from residual adhesive and taken from an area of the package that is free from any seams or folds. The test sample is cut to a width of 25.4 mm with a length that can accommodate a test span of 50.8 mm. The long side of the sample is parallel to the direction of interest (MD, CD). Normally in finished packages, the MD runs from the bottom to the top of the package, but this can be verified by determining the fiber orientation if in doubt. Ten replicate test samples should be prepared from the MD and ten additional replicates from the CD.

Program the tensile tester for a constant rate of extension uniaxial elongation to break as follows. Set the gauge length (test span) to 50.8 mm using a calibrated gauge block and zero the crosshead. Insert the test sample into the grips such that the long side is centered and parallel to the central pull axis of the tensile tester. Raise the crosshead at a rate of 508 mm/min until the test sample breaks, collecting force (N) and extension (mm) data at 100 Hz throughout the test. Construct a graph of force (N) versus extension (mm).

Read the maximum force (N) from the graph and record as Peak Force to the nearest 0.1 N, noting MD or CD. Read the extension at the maximum force (N) from the graph and record as Elongation at Break to the nearest 0.01 mm, noting MD or CD.

Calculate the arithmetic mean Peak Force for all MD replicates and then all CD replicates and record respectively as Mean MD Peak Force and Mean CD Peak Force to the nearest 0.1 N. Calculate the arithmetic mean Elongation at Break for all MD replicates and then all CD replicates and record respectively as Mean MD Elongation at Break and Mean CD Elongation at Break to the nearest 0.01 mm.

Tensile strength is calculated by dividing the Mean Peak Force (N) by the width of the test sample (25.4 mm). Calculate the tensile strength for the MD replicates and then the CD replicates and report respectively as MD Tensile Strength and CD Tensile Strength to the nearest 0.1 kN/m. Stretch at break is calculated by dividing the Mean Elongation at Break (mm) by the initial test length (test span) of 50.8 mm, and then multiplying by 100. Calculate the stretch at break for the MD replicates and then the CD replicates and report respectively as MD Stretch at Break and CD Stretch at Break to the nearest percent.

Web Modulus in MD (or MD Web Modulus): Take the force data (N) and divide by the width of the specimen, which in this case is 2.54 cm to obtain Force per cm. Take the extension data (mm), divide by the initial test length of 50.8 mm to obtain the strain (mm/mm). Construct a graph of Force per mm by strain. Calculate the slope of the curve between the 1%-2%, 2%-3%, 3%-4% and 4%-5% strain intervals and report the results in N/cm.

### Caliper

### Raw Material Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Caliper is measured with an electronic micrometer equipped with a pressure foot capable of exerting a steady pressure (±0.05 g/cm²) onto the test specimen, referred to as the test pressure. The test pressure is dependent on the type of material being tested. Table 10 below includes the test pressure for the various layers described herein. The micrometer type instrument with readings accurate to 0.003 mm. A suitable instrument is the 49 series micrometers available from Testing Machines Inc., or equivalent. The pressure foot is a smooth flat circular movable face with a diameter that is smaller than the test specimen and capable of exerting the required pressure. A suitable pressure foot has a diameter based on the test specimen; however, a smaller or larger foot can be used depending on the size of the specimen being measured. The pressure foot should be large enough to cover a meaningful area of the test specimen and be smaller than the size of the test specimen, such that the entire surface of the pressure foot engages the test specimen. Table 10 includes the size of the pressure foot diameter used to determine the caliper disclosed herein. The test specimen is supported by a horizontal flat specimen platform that is larger than and parallel to the surface of the pressure foot. The system is calibrated and operated per the manufacturer's instructions.

A test specimen is obtained by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, zero the micrometer, for example by pressing the zero button. Place the test specimen on the horizontal flat specimen platform. The specimen is to lay flat, free of wrinkles directly under the automatic cycling pressure foot ensuring the specimen will fully cover the area of contact between the pressure foot and the specimen platform. Press the test button. The pressure foot will descend and while the pressure foot rests on the test specimen, the test specimen's caliper will be measured. The caliper of the specimen thickness is then displayed on the digital read out. Record the caliper of the test specimen to the nearest 0.001 mm. In like fashion, the procedure is repeated until a total of ten replicate test samples have been measured. The arithmetic mean of the caliper measurements across all ten replicates is calculated and reported as Caliper to the nearest 0.001 mm.

TABLE 10

| Layer | Pressure foot Diameter | Test Pressure |
|---|---|---|
| Topsheet Layer | 56.4 mm | 0.5 kPa |
| Fluid Management Layer | 56.4 mm | 0.5 kPa |
| Core Layer | 50.0 mm | 0.5 kPa |
| First Barrier Layer | 16.0 mm | 50.4 kPa |
| Second Barrier Layer | 56.4 mm | 0.5 kPa |

### Finished Product Caliper

The caliper, or thickness, of an absorbent article test sample is measured as the distance between a reference platform on which the test sample rests and a pressure foot that exerts a specified amount of pressure onto the sample over a specified amount of time. All measurements are performed in a laboratory maintained at 23° C.±2° C. and 50%±2% relative humidity and test samples are conditioned in this environment for at least 2 hours prior to testing.

The caliper of the absorbent article test sample is measured using a manually operated micrometer equipped with a pressure foot capable of exerting a steady pressure of 6.33 g/cm²±0.05 g/cm² (0.62 kPa) onto the test location on the absorbent article test sample. The manually operated micrometer is a dead weight type instrument with readings accurate to 0.01 mm. A suitable instrument is Mitutoyo Series 543 ID-C Digimatic, available from VWR International, or equivalent. The pressure foot is a smooth flat circular movable face with a diameter that is smaller than the test location on the absorbent article test sample and capable of exerting the required pressure. A suitable pressure foot has a diameter of 40 mm; however, a smaller or larger foot can be used depending on the size of the test location on the test sample being measured. The test sample is supported by a horizontal flat reference platform that is larger than and parallel to the surface of the pressure foot. The system is calibrated and operated per the manufacturer's instructions.

The test sample is prepared by removing the absorbent article from any outer packaging, and if the article is folded, gently unfold it. The protective layer (i.e., release cover) covering the attachment adhesive is removed and a light coating of talc is applied to mitigate tackiness. The center of the test location is the intersection of the longitudinal and lateral midpoints on the body-facing side of the absorbent article. In like fashion, a total of five replicate test samples are prepared.

To measure caliper, the micrometer is zeroed against the horizontal flat reference platform. The test sample is then placed on the platform with the test location centered below the pressure foot. The pressure foot is gently lowered using a descent rate of 3.0 mm±1.0 mm per second until the full pressure is exerted onto the test sample. The full pressure is applied to the test sample for 5 seconds and then the caliper of the test sample is recorded to the nearest 0.001 mm. In like fashion, the procedure is repeated until a total of five replicate test samples have been measured. The arithmetic mean of the caliper measurements across all five replicates is calculated and reported as Caliper to the nearest 0.001 mm.

### Basis Weight

The basis weight of a test specimen is the mass (in grams) per unit area (in square meters) of a single layer of material and is measured in accordance with compendial method NWSP 130.1. The mass of the test specimen is cut to a known area, and the mass of the specimen is determined using an analytical balance accurate to 0.0001 grams. All measurements are performed in a laboratory maintained at 23° C.±2° C. and 50%±2% relative humidity and test specimens are conditioned in this environment for at least 2 hours prior to testing.

Measurements are made on test specimens obtained from rolls or sheets of the raw material. If raw materials are not available, test specimens are obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen must be as large as possible so that any inherent material variability is accounted for.

The dimensions of the single layer test specimen are measured using a calibrated steel metal ruler traceable to NIST, or equivalent. The are of the test specimen is calculated and recorded to the nearest 0.0001 square meter. An analytical balance is used to obtain the mass of the test specimen and the mass is recorded to the nearest 0.0001 gram. Basis weight is calculated by dividing the mass of the test specimen (in grams) by the area of the test specimen (square meters) and recorded to the nearest 0.01 grams per square meter (gsm). In like fashion, the procedure is repeated until a total of ten replicate test specimens have been measured. The arithmetic mean is calculated for the basis weight values recorded for all ten replicates and reported as Basis Weight to the nearest 0.01 grams/square meter.

Fiber Decitex (Dtex or dTex)

Textile webs (e.g., woven, nonwoven, airlaid) are comprised of individual fibers of material. Fibers are measured in terms of linear mass density reported in units of decitex. The decitex value is the mass in grams of a fiber present in 10,000 meters of that fiber. The decitex value of the fibers within a web of material is often reported by manufacturers as part of a specification. If the decitex value of the fiber is not known, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber. All testing is performed in a room maintained at a temperature of 23° C.±2° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is about 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The decitex dₖ value for each fiber type in the web material is calculated as follows:

$${d}_{k} = 10 \; 000 m \times {a}_{k} \times {ρ}_{k} \times 1 {0}^{- 6}$$

where dₖ is in units of grams (per calculated 10,000 meter length), aₖ is in units of μm², and ρₖ is in units of grams per cubic centimeter (g/cm³). Decitex is reported to the nearest 0.1 g (per calculated 10,000 meter length) along with the fiber type (e.g., PP, PET, cellulose, PP/PET bico).

### Combinations

In view of the foregoing disclosure, the following non-limiting combinations are contemplated.

A. An absorbent article comprising: a liquid pervious topsheet layer comprising cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises cellulosic material comprising fibers having from about 1.3 to about 10 decitex; a first barrier layer comprising at least one of cellulosic fibers and cellulosic film; a second barrier layer disposed adjacent to the first barrier layer, wherein the second barrier layer comprises cellulosic material; and an absorbent core layer disposed between the fluid management layer and the first barrier layer, wherein the absorbent article has an acquisition time of less than about 1 second and a wet through of less than about 0.3 g, and wherein the topsheet layer, the second barrier layer, the fluid management layer, the first barrier layer, and the absorbent core layer are void of synthetic fibers and films.

B. The absorbent article of paragraph A, wherein the topsheet layer has a topsheet basis weight of from about 25 to about 65 gsm, and wherein the cellulosic material comprises a plurality of fibers, wherein at least about 75% of the plurality of fibers has from about 0.9 to about 4.1 decitex.

C. The absorbent article according to any one of the preceding paragraphs, wherein the first barrier layer has a first surface and a second surface, wherein at least one of the first surface and the second surface comprises a plurality of ridges.

D. The absorbent article according to any one of the preceding paragraphs, wherein the second barrier layer has a second barrier layer acquisition time of greater than 3 seconds and a second barrier layer rewet of less than 1.5 g.

E. The absorbent article according to any one of the preceding paragraphs, wherein the absorbent core layer has a core basis weight from about 75 gsm to 230 gsm and a core caliper of from 0.35 mm to 5 mm.

F. The absorbent article according to paragraph E, wherein the absorbent article has an absorbent capacity of less than about 11 g.

G. The absorbent article according to any one of the preceding paragraphs, wherein the cellulosic material of the topsheet layer comprises at least one of rayon and cotton.

H. The absorbent article according to paragraph G, wherein the cellulosic material comprises at least one of hydrophobic viscose and hydrophilic viscose.

I. The absorbent article according to any one of the preceding paragraphs, comprising an adhesive, wherein the adhesive is substantially free of or void of synthetic polymers.

J. The absorbent article according to any one of the preceding paragraphs, wherein the absorbent core layer comprises a first stratum of cellulosic material and a second stratum of cellulosic material.

K. The absorbent article according to paragraph J, wherein the first stratum of cellulosic material comprises pulp fibers and the second stratum of cellulosic material comprises staple fibers.

L. The absorbent article according to paragraph A, wherein the absorbent core layer comprises a first stratum of cellulosic material, and wherein the first stratum of cellulosic material comprises pulp fibers, and wherein the pulp fibers are compressed to form hydrogen bonds.

M. The absorbent article according to paragraph A, wherein the absorbent core layer comprises a stratum layer of cellulosic material, and wherein the first stratum of cellulosic material comprises pulp fibers, and wherein the pulp fibers are at least partially joined using a binder.

N. The absorbent article according to paragraph M, wherein the binder is substantially free of or void of synthetic polymers.

A1. An absorbent article comprising: a liquid pervious topsheet layer comprising a cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising carded staple fibers, wherein at least 50% of the carded staple fibers have from about 3.3 to about 10 decitex, and wherein the fluid management layer has a weighted average area moment of inertia of from about 2,000 μm⁴ to 95,000 μm⁴; a second barrier layer adjacent to the fluid management layer, wherein the second barrier layer comprises a cellulosic material; and an absorbent core layer disposed between the fluid management layer and the second barrier layer, wherein the absorbent article is substantially free of synthetic fibers and films.

B1. The absorbent article of according to paragraph A1, wherein the absorbent article has a rewet of less than about 0.2 g and acquisition time is less than about 1 second.

C1. The absorbent article according to any one of the preceding paragraphs, comprising a first barrier layer disposed between the absorbent core layer and the second barrier layer, wherein the first barrier layer comprises at least one of cellulosic fibers and cellulosic film.

D1. The absorbent article according to any one of the preceding paragraphs, wherein the staple fibers comprise a first plurality of fibers and a second plurality of fibers, wherein the first plurality of fibers have a first area moment of inertia and the second plurality of fibers have a second area moment of inertia, wherein the first area moment of inertia and the second area moment of inertia are different.

E1. The absorbent article according to paragraph D1, wherein the weighted average of the first area moment of inertia and the second area moment of inertia is from about 7,000 μm⁴ to 25,000 μm⁴.

F1. The absorbent article according to any one of the preceding paragraphs, wherein the staple fibers of the fluid management layer are entangled by at least one of spunlacing and needlepunching the staple fibers.

G1. The absorbent article according to any one of the preceding paragraphs, wherein the staple fibers of the fluid management layer have a non-circular cross section.

H1. The absorbent article according to paragraph A1, wherein the staple fibers of the fluid management layer comprise a first plurality of staple fibers having a first cross-sectional shape and a second plurality of staple fibers having a second cross-sectional shape, and wherein the first cross-sectional shape and the second cross-sectional shape are different.

I1. The absorbent article according to any one of the preceding paragraphs, wherein the fluid management layer is void of synthetic fibers and films.

J1. The absorbent article according to paragraph I1, wherein at least one of the topsheet layer, the second barrier layer, first barrier layer, and the absorbent core layer is void of synthetic fibers and films.

A2. An absorbent article comprising: a liquid pervious topsheet layer comprising a cellulosic material; a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising fibers having from about 1.7 to about 10 decitex; a first barrier layer comprising at least one of cellulosic fibers and cellulosic film, wherein the first barrier layer has a repellency of at least 5 seconds and a 10% of Pore less than about 20 microns; a second barrier layer adjacent to the first barrier layer, wherein the second barrier layer comprises cellulosic material; and an absorbent core layer disposed between the fluid management layer and the first barrier layer, wherein the first barrier layer and the fluid management layer are void of synthetic fibers and films.

B2. The absorbent article according to paragraph A2, wherein the absorbent article has a Wet Through of less than about 0.3 g.

C2. The absorbent article according to any one of the preceding paragraphs, wherein the first barrier layer has a repellency of greater than 10 seconds.

D2. The absorbent article according to any one of the preceding paragraphs, wherein the first barrier layer comprises a hydrophobic coating on at least one of a first surface and a second surface.

E2. The absorbent article according to any one of the preceding paragraphs, wherein the first barrier layer comprises at least 50% of Northern softwood fibers.

F2. The absorbent article of according to any one of the preceding paragraphs, wherein the first barrier layer has a dry burst strength of at least 100 kPa and a wet burst strength of at least 14 kPa.

G2. The absorbent article of according to any one of the preceding paragraphs, wherein the first barrier layer has a basis weight of from about 45 to about 65 gsm.

H2. The absorbent article of according to any one of the preceding paragraphs, wherein the first barrier layer has a machine direction modulus of less than about 550 N/cm.

I2. The absorbent article of according to any one of the preceding paragraphs, wherein the first barrier layer material is creped paper.

J2. The absorbent article of according to any one of the preceding paragraphs, wherein the first barrier layer is a creped paper comprising a plurality of ridges, wherein the first barrier layer is configured to stretch in a stretch direction perpendicular to a ridge direction of the plurality of ridges.

K2. The absorbent article according to any one of the preceding paragraphs, wherein the first barrier layer has an unstretched length and a stretched length, and wherein the stretched length is at least 10% greater than the unstretched length.

L2. The absorbent article according to any one of the preceding paragraphs, wherein the cellulosic fibers of the fluid management layer are pulp fibers.

M2. The absorbent article of according to paragraph L2, wherein the pulp fibers comprise at least one of softwood fibers, hardwood fibers, bamboo fibers, hemp fibers, and eucalyptus fibers.

N2. The absorbent article according to any one of the preceding paragraphs, wherein the absorbent article is void of synthetic fibers and films.

O2. The absorbent article according to paragraph A2, wherein at least one of the topsheet layer, the second barrier layer, the fluid management layer, and the absorbent core layer are void of synthetic fibers and films.

P2. The absorbent article according to any one of the preceding paragraphs, wherein the second barrier layer has a second barrier basis weight of from about 30 to about 65 gsm and a second barrier caliper of from about 0.15 to about 1.0 mm at 0.5 kPa.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Further, for any range of values specifically recited herein, all values within the recited ranges and any ranges created thereby are deemed to be recited such that any range between or including the recited values of the range herein are to be considered disclosed and may be recited in one or more claims.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a liquid pervious topsheet layer comprising cellulosic material;
a fluid management layer disposed adjacent to the topsheet layer, the fluid management layer having a basis weight of from about 45 to 75 gsm and a caliper of from about 0.5 to 1.6 mm, wherein the fluid management layer comprises cellulosic material comprising fibers having from about 1.3 to about 10 decitex;
a first barrier layer comprising at least one of cellulosic fibers and cellulosic film;
a second barrier layer disposed adjacent to the first barrier layer, wherein the second barrier layer comprises cellulosic material; and
an absorbent core layer disposed between the fluid management layer and the first barrier layer,
wherein the absorbent article has an acquisition time of less than about 1 second and a wet through of less than about 0.3 g, and
wherein the topsheet layer, the second barrier layer, the fluid management layer, the first barrier layer, and the absorbent core layer are void of synthetic fibers and films.

2. The absorbent article of claim 1, wherein the topsheet layer has a topsheet basis weight of from about 25 to about 65 gsm, and wherein the cellulosic material comprises a plurality of fibers, wherein at least about 75% of the plurality of fibers has from about 0.9 to about 4.1 decitex.

3. The absorbent article of claim 1, wherein the first barrier layer has a first surface and a second surface, wherein at least one of the first surface and the second surface comprises a plurality of ridges.

4. The absorbent article of claim 1, wherein the second barrier layer has a second barrier layer acquisition time of greater than 3 seconds and a second barrier layer rewet of less than 1.5 g.

5. The absorbent article of claim 1, wherein the absorbent core layer has a core basis weight from about 75 gsm to 230 gsm and a core caliper of from 0.35 mm to 5 mm.

6. The absorbent article of claim 5, wherein the absorbent article has an absorbent capacity of less than about 11 g.

7. The absorbent article of claim 1, wherein the cellulosic material of the topsheet layer comprises at least one of rayon and cotton.

8. The absorbent article of claim 7, wherein the cellulosic material comprises at least one of hydrophobic viscose and hydrophilic viscose.

9. The absorbent article of claim 1, comprising an adhesive, wherein the adhesive is substantially free of or void of synthetic polymers.

10. The absorbent article of claim 1, wherein the absorbent core layer comprises a first stratum of cellulosic material and a second stratum of cellulosic material.

11. The absorbent article of claim 1, wherein the absorbent core layer comprises a stratum layer of cellulosic material, and wherein the first stratum of cellulosic material comprises pulp fibers, and wherein the pulp fibers are at least partially joined using a binder.

12. The absorbent article of claim 11, wherein the binder is substantially free of or void of synthetic polymers.

13. An absorbent article comprising:
a liquid pervious topsheet layer comprising a cellulosic material;
a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising carded staple fibers, wherein at least 50% of the carded staple fibers have from about 3.3 to about 10 decitex, and wherein the fluid management layer has a weighted average area moment of inertia of from about 2,000 μm⁴ to 95,000 μm⁴;
a second barrier layer adjacent to the fluid management layer, wherein the second barrier layer comprises a cellulosic material; and
an absorbent core layer disposed between the fluid management layer and the second barrier layer,
wherein the absorbent article is substantially free of synthetic fibers and films.

14. The absorbent article of claim 13, wherein the absorbent article has a rewet of less than about 0.2 g and acquisition time is less than about 1 second.

15. The absorbent article of claim 13, wherein the staple fibers comprise a first plurality of fibers and a second plurality of fibers, wherein the first plurality of fibers have a first area moment of inertia and the second plurality of fibers have a second area moment of inertia, wherein the first area moment of inertia and the second area moment of inertia are different.

16. The absorbent article of claim 15, wherein the weighted average of the first area moment of inertia and the second area moment of inertia is from about 7,000 μm⁴ to 25,000 μm⁴.

17. The absorbent article of claim 13, wherein the staple fibers of the fluid management layer comprise a first plurality of staple fibers having a first cross-sectional shape and a second plurality of staple fibers having a second cross-sectional shape, and wherein the first cross-sectional shape and the second cross-sectional shape are different.

18. The absorbent article of claim 13, wherein the fluid management layer is void of synthetic fibers and films.

19. An absorbent article comprising:
a liquid pervious topsheet layer comprising a cellulosic material;
a fluid management layer disposed adjacent to the topsheet layer, the fluid management layer having a basis weight of from about 45 to 75 gsm and a caliper of from about 0.5 to 1.6 mm, wherein the fluid management layer comprises a cellulosic material comprising fibers having from about 1.7 to about 10 decitex;
a first barrier layer comprising at least one of cellulosic fibers and cellulosic film, wherein the first barrier layer has a repellency of at least 5 seconds and a 10% of Pore less than about 20 microns;
a second barrier layer adjacent to the first barrier layer, wherein the second barrier layer comprises cellulosic material; and
an absorbent core layer disposed between the fluid management layer and the first barrier layer,
wherein the first barrier layer and the fluid management layer are void of synthetic fibers and films.

20. The absorbent article of claim 19, wherein the first barrier layer has a dry burst strength of at least 100 kPa and a wet burst strength of at least 14 kPa.

21. The absorbent article of claim 19, wherein the first barrier layer has a machine direction modulus of less than about 550 N/cm.

22. The absorbent article of claim 19, wherein the first barrier layer material is creped paper, wherein the creped paper comprising a plurality of ridges, wherein the first barrier layer is configured to stretch in a stretch direction perpendicular to a ridge direction of the plurality of ridges.

23. The absorbent article of claim 19, wherein the first barrier layer has an unstretched length and a stretched length, and wherein the stretched length is at least 10% greater than the unstretched length.

24. The absorbent article of claim 19, wherein the absorbent article is void of synthetic fibers and films.

25. The absorbent article of claim 19, wherein at least one of the topsheet layer, the second barrier layer, the fluid management layer, and the absorbent core layer are void of synthetic fibers and films.
